(19) United States
(12) Patent Application Publication
(10) Pub. No.: US 2016/0007916 A1
(43) Pub. Date: Jan. 14, 2016

(54) BIOLOGICAL INFORMATION DETECTING DEVICE

(71) Applicant: Seiko Epson Corporation, Tokyo (JP)

(72) Inventor: Takanori IWAWAKI, Hachiouji-shi (JP)

(21) Appl. No.: 14/790,499

(22) Filed: Jul. 2, 2015

(30) Foreign Application Priority Data
Jul. 10, 2014 (JP) 2014-141989

(51) Int. Cl. A61B 5/00; A61B 5/0205

(52) U.S. Cl. CPC A61B 5/4812; A61B 5/0059; A61B 5/4809; A61B 5/7214; A61B 5/4035; A61B 5/0205; A61B 5/02427

(57) ABSTRACT

A biological information detecting device includes a first light reception unit which receives light from a subject, a second light reception unit which receives light from the subject, at least one light emission unit which emits light to the subject, and a processing unit. When a distance between the light emission unit and the first light reception unit is L1, and a distance between the light emission unit and the second light reception unit is L2, L1<L2 is satisfied. The processing unit determines an active state of the subject on the basis of a first detection signal detected by the first light reception unit and a second detection signal detected by the second light reception unit, and controls the biological information detecting device according to the active state.

This application claims priority to Japanese Patent Application No. 2014-141989, filed Jul. 10, 2014, the entirety of which is hereby incorporated by reference.

### BACKGROUND

1. Technical Field

The present invention relates to a biological information detecting device or the like.

2. Related Art

An object of a device for measuring biological information such as a pulse wave, for example, is to promote health or diet, or is to manage quality of sleep or sleep disorders by monitoring a sleep state. For example, in JP-A-2001-61819, a technology is disclosed in which an awake state or a sleep state is detected, the amount of time to fall asleep or the amount of time to deep sleep, the number of times of awakening during sleep (awakening without consciousness), and the like are obtained from a detection result, and advice for improving sleep is offered to a user on the basis of the content of the obtained information.

Such a biological information detecting device, for example, is usually portable. In such a portable device, reduction in size and weight is required, and thus there is a limit in capacity of a battery, and low power consumption is required.

Incidentally, when the biological information such as a pulse wave is measured, a noise (a body motion noise) due to motion of a body other than the biological information is added to a detection signal. As a method of reducing the body motion noise, a method is considered in which two photoelectric sensors are used, and the body motion noise is mainly detected by one photoelectric sensor. However, the two photoelectric sensors are used, and thus power consumption increases.

Furthermore, in JP-A-2001-61819 described above, a technical problem of low power consumption in the biological information detecting device, and a solving method thereof are not disclosed. In addition, a specific determining method of an active state (for example, an awake state, a sleep state, or the like), and a specific measuring method of the biological information are not disclosed.

### SUMMARY

An advantage of some aspects of the invention is to provide a biological information detecting device or the like in which low power consumption is able to be realized according to an active state of a subject (a user).

An aspect of the invention relates to a biological information detecting device including a first light reception unit which receives light from a subject; a second light reception unit which receives light from the subject; at least one light emission unit which emits light to the subject; and a processing unit, in which when a distance between the light emission unit and the first light reception unit is L1, and a distance between the light emission unit and the second light reception unit is L2, L1<L2 is satisfied, and the processing unit determines an active state of the subject on the basis of a first detection signal detected by the first light reception unit and a second detection signal detected by the second light reception unit, and controls a first detection operation of the light emission unit and the first light reception unit and a second detection operation of the light emission unit and the second light reception unit according to the active state.

According to the aspect of the invention, the active state of the subject is determined on the basis of the first detection signal and the second detection signal, and the first detection operation of the light emission unit and the first light reception unit and the second detection operation of the light emission unit and the second light reception unit are controlled according to the active state. Accordingly, for example, the on and off or the like of the detection operation, for example, is able to be adaptively controlled according to the active state such as an awake state or a sleep state, and low power consumption is able to be realized according to the active state.

In the aspect of the invention, the active state may be a sleep state of the subject, and the processing unit may set the second detection operation to be in a normal operation mode when it is determined that the subject is in a first sleep state, and may set the second detection operation to be in a non-operation mode when it is determined that the subject is in a second sleep state which is deeper than the first sleep state.

With this configuration, the detection operation is able to be controlled in the operation mode which is different in the first sleep state and the second sleep state in the sleep state. That is, in the second sleep state which is a deeper sleep state, it is considered that body motion occurs less than that in the first sleep state, and thus it is possible to realize low power consumption by setting the second detection operation to be in the non-operation mode. In addition, in the first sleep state which is a shallower sleep state, it is possible to reduce a body motion noise using the second detection signal by setting the second detection operation to be in the normal operation mode.

In the aspect of the invention, the processing unit may allow the light emission unit to emit light during a first period of detecting the light from the subject by the first light reception unit and a second period of detecting the light from the subject by the second light reception unit in the normal operation mode of the second detection operation, and may stop light emission of the light emission unit during the second period in the non-operation mode of the second detection operation.

As described above, in the normal operation mode, the light emission unit emits light and performs the detection operation in different periods of the first detection operation and the second detection operation. In contrast, in the non-operation mode of the second detection operation, only the first detection operation is performed, and thus the light emission of the light emission unit is stopped at a timing corresponding to the second detection operation. Accordingly, in a predetermined sleep state, the number of times of the light emission of the light emission unit is decreased by half, and it is possible to reduce power consumption in the light emission unit.

In the aspect of the invention, the first sleep state may be REM sleep, and the second sleep state may be non-REM sleep.

With this configuration, the detection operation is able to be controlled in the operation mode which is in the REM sleep and the non-REM sleep in the sleep state. That is, in the non-REM sleep, the second detection operation is set to be in the non-operation mode, and thus it is possible to realize low power consumption. In addition, in the REM sleep, the second detection operation is set to be in the normal operation mode, and thus it is possible to reduce a body motion noise using the second detection signal.

In the aspect of the invention, the processing unit may set the second detection operation to be in a normal operation mode when it is determined that the subject is in an awake state, and may set the second detection operation to be in a non-operation mode when it is determined that the subject is in a predetermined sleep state.

With this configuration, in the awake state where the amount of activity increases and a body motion noise is easily generated, the body motion noise from the first detection signal is able to be reduced by using the second detection signal, and high-precision biological information is able to be detected. Then, in a predetermined sleep state where the amount of activity decreases and a body motion noise is rarely generated, the second detection operation is stopped, and thus low power consumption is able to be realized.

In the aspect of the invention, the processing unit may perform body motion noise reduction processing which reduces a body motion noise of the first detection signal on the basis of the second detection signal, and may calculate biological information on the basis of the first detection signal after being subjected to the body motion noise reduction processing.

The first light reception unit and the second light reception unit are included, and the distances L1 and L2 from the light emission unit are different in the first light reception unit and the second light reception unit, and thus it is possible to make sensitivity with respect to the biological information and the body motion different in each of the light reception units. Accordingly, the biological information is able to be mainly detected by the first light reception unit, and the body motion noise is able to be mainly detected by the second light reception unit, and thus the body motion noise from the first detection signal is reduced by using the second detection signal, and high-precision biological information is able to be detected.

In the aspect of the invention, the processing unit may obtain pulse wave information as the biological information, and may determine the active state on the basis of the pulse wave information.

The pulse wave information is associated with an activity balance of an automatic nerve, and the activity balance of the automatic nerve is changed according to the active state. That is, the pulse wave information is obtained as the biological information, and thus the active state is able to be determined.

In the aspect of the invention, the processing unit may obtain a first index indicating activity of a sympathetic nerve and a second index indicating activity of a parasympathetic nerve by frequency analysis of the pulse wave information, and may determine the active state on the basis of the first index and the second index.

The pulse wave information is subjected to the frequency analysis, and thus frequency properties of the pulse wave are able to be acquired. In the frequency properties, not only a beat frequency but also a fluctuation frequency of the beat frequency is included. In the fluctuation, information of the activity balance of the automatic nerve is included, and thus the active state is able to be determined by obtaining the fluctuation as the first index and the second index.

In the aspect of the invention, a motion sensor unit which detects body motion information of the subject may be further included, in which the processing unit may determine the active state on the basis of the body motion information.

In the aspect of the invention, the processing unit may set the motion sensor unit to be in a low power consumption mode when it is determined that the subject has transitioned from the awake state to the sleep state.

The body motion decreases after initiation of sleep, and thus it is not necessary that the motion sensor unit performs the same operation as that in the awake state. For this reason, when it is determined as the initiation of sleep, the motion sensor unit is set to be in the low power consumption mode, and thus the number of times of acquisition of the body motion information is able to be reduced, and power consumption in the motion sensor unit is able to be reduced.

Another aspect of the invention relates to a biological information detecting device including a first light reception unit which receives light from a subject; a second light reception unit which receives light from the subject; at least one light emission unit which emits light to the subject; a substrate on which at least the first light reception unit and the light emission unit are arranged; a light transmissive member which is disposed in a position on the subject side from the first light reception unit side and the second light reception unit side, transmits the light from the subject, and is in contact with the subject at the time of measuring biological information of the subject; and a processing unit; in which in a plan view of a direction from the biological information detecting device to the subject, when a distance between the substrate and a surface of the light transmissive member in contact with the subject in a region in which the light transmissive member and the first light reception unit overlap each other is h1, and a distance between the substrate and a surface of the light transmissive member in contact with the subject in a region in which the light transmissive member and the second light reception unit overlap each other is h2, h1>h2 may be satisfied, and the processing unit may determine an active state of the subject on the basis of a first detection signal detected by the first light reception unit and a second detection signal detected by the second light reception unit, and may control a first detection operation of the light emission unit and the first light reception unit and a second detection operation of the light emission unit and the second light reception unit according to the active state.

### BRIEF DESCRIPTION OF THE DRAWINGS

The invention will be described with reference to the accompanying drawings, wherein like numbers reference like elements.

FIG. 1A is an external view of a biological information detecting device, and FIG. 1B is an external view of the biological information detecting device and an explanatory diagram for mounting of the biological information detecting device and communication with a terminal device.

FIG. 2 is a functional block diagram of the biological information detecting device.

FIGS. 3A to 3C are diagrams illustrating an LF component and an HF component of a heart rate.

FIG. 4A is a diagram schematically illustrating a relationship between LF/HF and an active state, and FIG. 4B is a diagram schematically illustrating a relationship between HF/(LF+HF) and the active state.

FIG. 5 is a diagram illustrating a determining method of an awake state and a sleep state, and control of a detection operation in each state.

FIG. 6 is an example of a connection configuration of the biological information detecting device.

FIG. 7 is a flowchart of determining the active state and of controlling the detection operation.

FIG. 8 is a specific flowchart of initiation of sleep determination processing.

FIG. 9 is a diagram illustrating vibration factors.

FIG. 10 is a specific flowchart of sleep state determination processing.

FIG. 11 is a timing chart of a light emission operation in each operation mode.

FIG. 12 is an operation timing chart of a photoelectric sensor and an acceleration sensor in each operation mode.

FIG. 13 is a specific flowchart of awakening determination processing.

FIGS. 14A and 14B are a sectional view and a plan view illustrating an example of an arrangement of a light emission unit and a light reception unit, and an example of a configuration of a light transmissive member.

FIG. 15 is a diagram illustrating an influence of a distance between the light emission unit and the light reception unit on a penetration depth of light.

FIG. 16 is a diagram illustrating a relationship of the distance between the light emission unit and the light reception unit, and a signal intensity of a detection signal.

FIG. 17 is a diagram exemplifying a change in absorbancy with respect to a pressing force.

FIG. 18 is a diagram exemplifying a change in body motion noise sensitivity with respect to a pressing force.

FIGS. 19A and 19B are diagrams illustrating body motion noise reduction processing by using spectral subtraction.

FIG. 20 is a diagram illustrating the body motion noise reduction processing by using adaptive filter processing.

FIG. 21 is a diagram illustrating a flow of signal processing.

### DESCRIPTION OF EXEMPLARY EMBODIMENTS

Hereinafter, preferred embodiments of the invention will be described in detail. Furthermore, this embodiment described hereinafter does not limit the aspects of the invention, the entire configuration to be described in this embodiment is not essential as a solving method of the invention.

### 1. Biological Information Detecting Device

Hereinafter, a biological information detecting device of this embodiment will be described. Furthermore, hereinafter, a case where a pulse wave (the number of pulses) is measured as biological information will be described as an example, but this embodiment is not limited thereto, and is able to be applied to a case where biological information other than the pulse wave (for example, oxygen saturation in blood, body temperature, a state of peripheral blood circulation, heart rate, and the like) is detected.

The pulse wave which is the biological information appears as a change in volume of blood. The change in the volume of the blood (a change in blood volume of a portion which is a measurement target) is captured by a photoelectric sensor, and thus the pulse wave is able to be measured. However, the volume of the blood in the portion to be measured is also changed according to motion of a human body (hereinafter, referred to as body motion) in addition to a heart beat (that is, the pulse wave). For this reason, when the pulse wave is measured by the photoelectric sensor, noise due to the body motion may be included in a pulsation while the blood flows from a heart to the portion to be measured. That is, the blood is fluid, and a blood vessel has elasticity, and thus the flow of the blood due to the body motion generates a change in the blood volume, and may be measured as a false pulse beat.

As a method of reducing such a body motion noise, a method is included in which a component corresponding to a pulse signal among detection signals of the photoelectric sensor is maintained as much as possible, and a component corresponding to the body motion noise is reduced (in a restricted sense, eliminated). In reduction processing of the body motion noise, it is necessary to understand the signal component corresponding to the body motion noise.

In this embodiment, a second light reception unit sets sensitivity of the pulse signal to be low and sensitivity of the body motion noise to be high by using a fact that the body motion noise is included in the detection signal of the photoelectric sensor, and thus the detection signal including the body motion noise is able to be mainly acquired. In the second light reception unit, when a signal corresponding to the body motion noise is able to be detected, a component corresponding to a detection signal of the second light reception unit is eliminated (reduced) from a detection signal of a first light reception unit, and thus the body motion noise is able to be reduced.

When such a noise reduction is performed, in an activity time zone (an awake state) of daily life, it is necessary to constantly operate the first light reception unit and the second light reception unit in order to efficiently eliminate the body motion noise. However, the second light reception unit for eliminating the body motion noise is operated, and thus an operation duration time (that is, power consumption) is shortened, compared to a case where the pulse wave is acquired by only the first light reception unit mainly detecting the pulse wave.

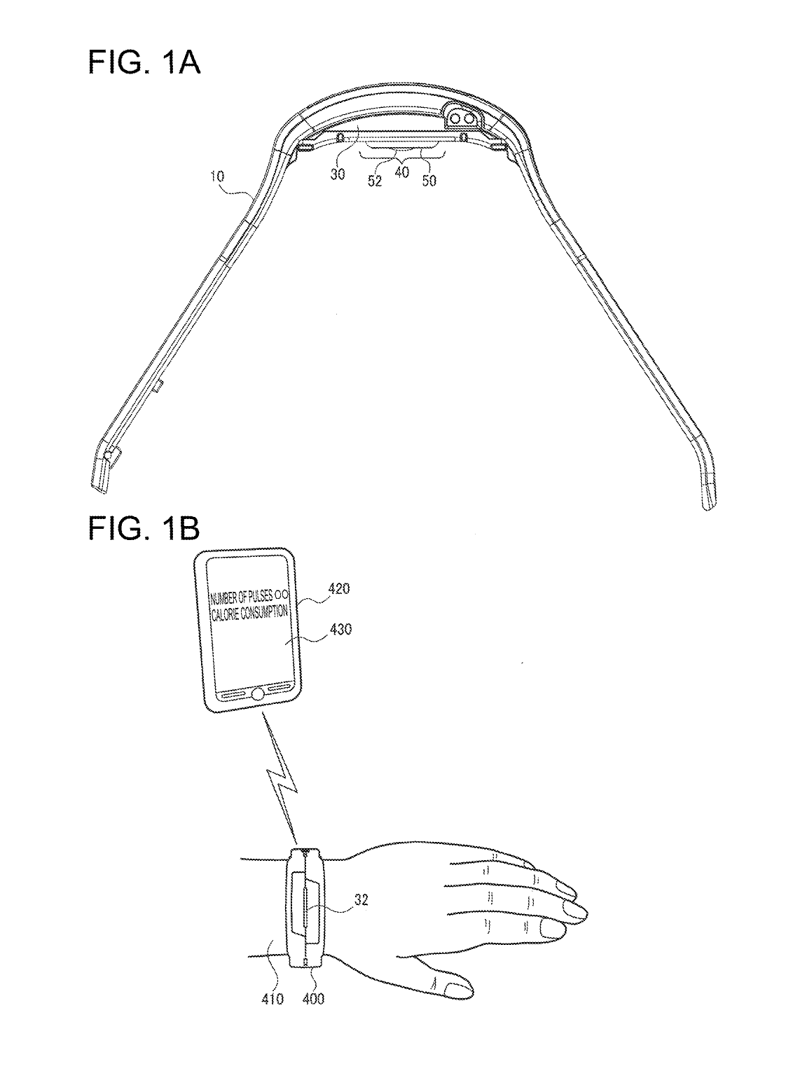

In FIG. 1A and FIG. 1B, an external view of a biological information detecting device (a biological information measuring device) of this embodiment which is able to solve such a problem is illustrated.

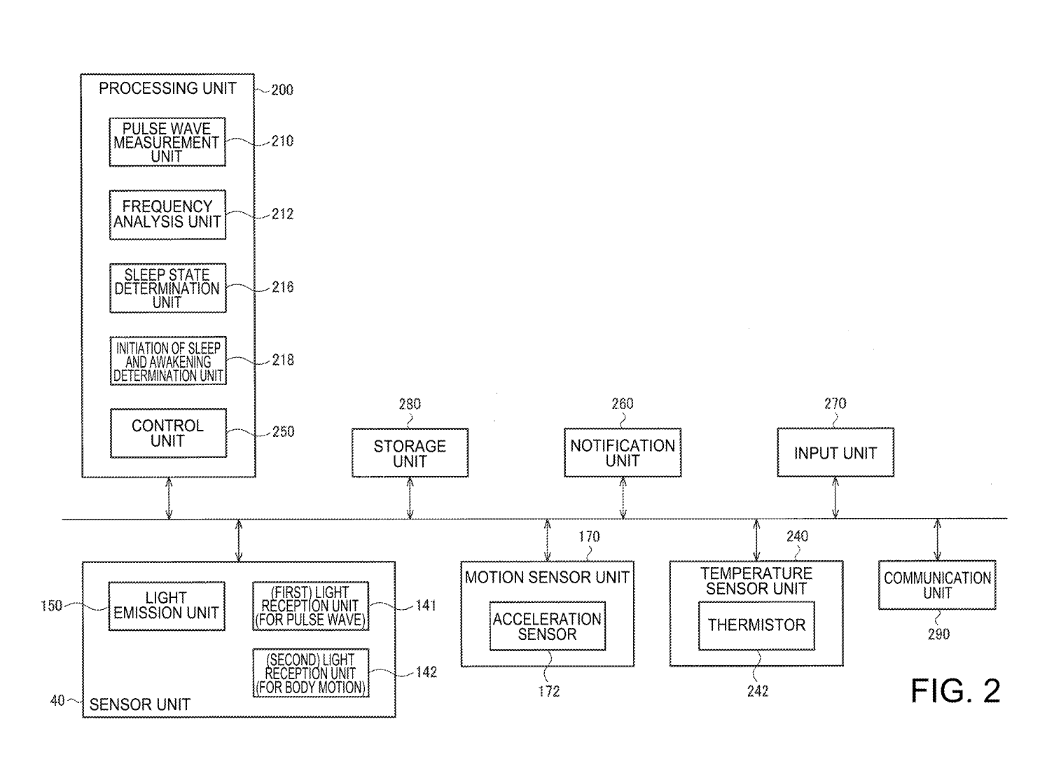

As illustrated in FIG. 1A, the biological information detecting device includes a band portion 10, a case portion 30, and a sensor unit 40. The case portion 30 is attached to the band portion 10. The sensor unit 40 is disposed in the case portion 30. In addition, the biological information detecting device includes a processing unit 200 which is described later and is illustrated in FIG. 2. The processing unit 200 is disposed in the case portion 30, and detects a pulse wave (biological information) on the basis of a detection signal from the sensor unit 40.

The band portion 10 is wound around a wrist of a user in order to mount the biological information detecting device thereon. In the band portion, a band hole and buckle portion, not illustrated, are disposed. The amount of a pressing force of the sensor unit 40 (a pressure pressed to a wrist surface) is adjusted according to which band hole is inserted with a projection portion of the buckle portion.

The case portion 30 corresponds to a main body portion (a case) of the biological information detecting device. Various constituents of the biological information detecting device such as the sensor unit 40, and the processing unit 200 are disposed inside the case portion 30.

As illustrated in FIG. 1B, a light emission window portion 32 formed of a light transmissive member is disposed in the case portion 30. Light from the light emission unit (an LED, a light emission unit for a notification different from a light emission unit 150 of the sensor unit 40) which is disposed in the case portion 30 is emitted to the outside of the case portion 30 through the light emission window portion 32.

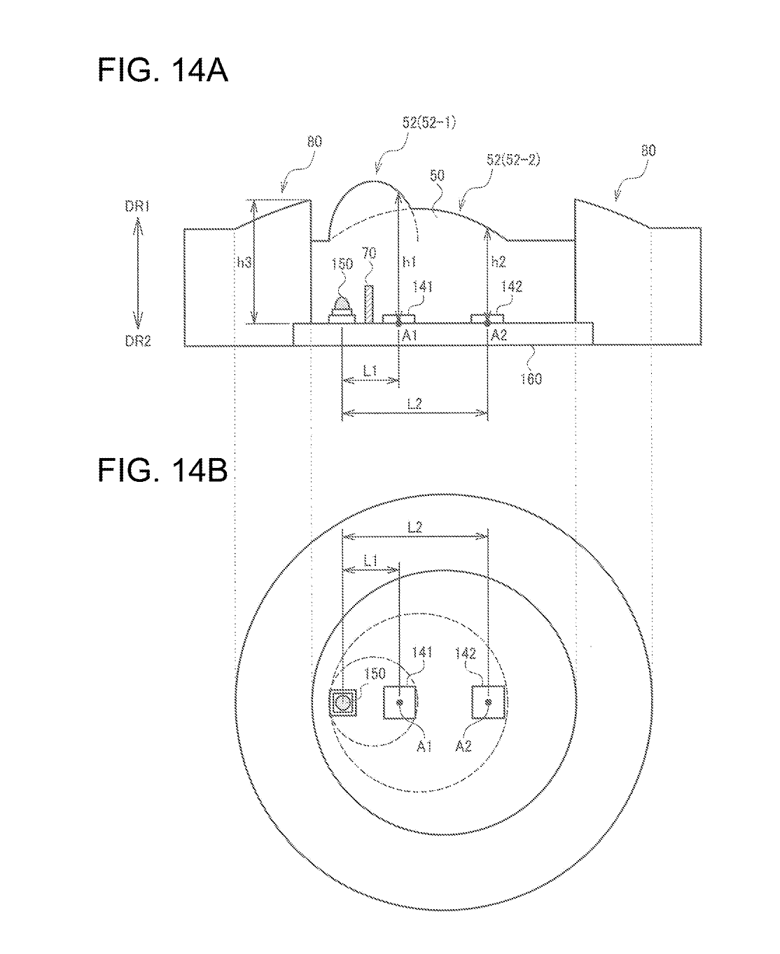

The sensor unit 40 detects the pulse wave of the user. For example, as illustrated in FIG. 14A described later, the sensor unit 40 includes a first light reception unit 141, a second light reception unit 142, and the light emission unit 150. In addition, the sensor unit 40 is formed of a light transmissive member 50, and includes a convex portion 52 which applies a pressing force by being in contact with a skin surface of a subject. Thus, in a state where the convex portion 52 applies a pressing force to the skin surface, the light emission unit 150 emits light, each of the first light reception unit 141 and the second light reception unit 142 receives light reflected on the subject (a blood vessel), and a light reception result thereof is output to the processing unit 200 as a first detection signal and a second detection signal. Then, the processing unit 200 performs noise reduction processing with respect to the first detection signal on the basis of the second detection signal of the sensor unit 40, and detects the pulse wave on the basis of the first detection signal after being subjected to the noise reduction processing.

Mounting of a biological information detecting device 400 and communication with a terminal device 420 will be described with reference to FIG. 1B.

As illustrated in FIG. 1B, the user mounts the biological information detecting device 400 on a wrist 410 as similar to a watch. As described above, the convex portion 52 of the sensor unit 40 applies a pressing force by being in contact with the skin surface of the wrist 410, and in this state, the pulse wave is detected.

The biological information detecting device 400 and the terminal device 420 are connected for communication, and are able to perform data communication. The terminal device 420, for example, a portable communication terminal such as a smart phone, a mobile phone, and a feature phone, or an information processing terminal such as a tablet type computer. As the communication connection, for example, near-field wireless communication such as Bluetooth (registered trademark) is able to be adopted. On a display unit 430 (an LCD or the like) of the terminal device 420, various information items (for example, the number of pulses, calorie consumption, or the like) obtained on the basis of the detection signal of the sensor unit 40 are able to be displayed. Furthermore, calculation processing of the information such as the number of pulses or the calorie consumption may be performed in the biological information detecting device 400, or at least a part thereof may be performed in the terminal device 420.

The light emission window portion 32 is disposed in the biological information detecting device 400, and the various information items are notified to the user by light emission of the light emission unit for a notification (illuminating and blinking). For example, at the time of getting into a fat combustion zone or getting out of the fat combustion zone, this is notified by the light emission of the light emission unit through the light emission window portion 32. Alternatively, when mail or the like is received in the terminal device 420, this is notified to the biological information detecting device 400 from the terminal device 420, and the light emission unit of the biological information detecting device 400 emits the light, and thus the reception of the mail or the like is notified to the user.

Thus, in FIG. 1B, the display unit is not disposed in the biological information detecting device 400, and information which is required to be notified in letters and figures is displayed on the display unit 430 of the terminal device 420. Furthermore, this embodiment is not limited thereto, and the display unit may be disposed in the biological information detecting device 400.

In FIG. 2, a functional block diagram of the biological information detecting device is illustrated. The biological information detecting device includes the sensor unit 40, a motion sensor unit 170, the processing unit 200, a temperature sensor unit 240, a notification unit 260, an input unit 270 (an operation unit), a storage unit 280, and a communication unit 290.

The sensor unit 40 detects the pulse wave, and includes a first light reception unit 141, a second light reception unit 142, and the light emission unit 150. Furthermore, in FIG. 2, an example in which the light emission unit 150 is shared by a plurality of light reception units is illustrated, but the number of light emission units is not limited to one, and two or more light emission units may be disposed.

A pulse wave sensor (a photoelectric sensor) is realized by the first light reception unit 141, the second light reception unit 142, and the light emission unit 150. That is, a first pulse wave sensor is realized by the first light reception unit 141 and the light emission unit 150, and a second pulse wave sensor is realized by the second light reception unit 142 and the light emission unit 150. The sensor unit 40 outputs a signal detected by a plurality of pulse wave sensors as the detection signal (a pulse wave detection signal).

The motion sensor unit 170 outputs a body motion detection signal which is a signal changed according to the body motion, on the basis of sensor information of various motion sensors. The motion sensor unit 170, for example, includes an acceleration sensor 172 as the motion sensor. Furthermore, the motion sensor unit 170 may include a pressure sensor, a gyro sensor, and the like as the motion sensor.

The temperature sensor unit 240 outputs a temperature detection signal which is changed according to a body temperature, on the basis of sensor information of various temperature sensors. The temperature sensor unit 240, for example, includes a thermistor 242 as the temperature sensor. Furthermore, the temperature sensor unit 240 may include a thermocouple, or the like as the temperature sensor.

The processing unit 200, for example, performs various signal processings and control processings by using the storage unit 280 as a working region, and for example, is able to be realized by a processor such as a CPU or a logic circuit such as an ASIC. The processing unit 200 includes a pulse wave measurement unit 210, a frequency analysis unit 212, a sleep state determination unit 216, an initiation of sleep and awakening determination unit 218, and a control unit 250.

The pulse wave measurement unit 210 performs signal processing with respect to the pulse wave detection signal from the sensor unit 40, the body motion detection signal from the motion sensor unit 170, or the like, and calculates beat information from the signals after being subjected to the signal processing. The beat information, for example, is information such as the number of pulses. Specifically, the pulse wave measurement unit 210 performs body motion noise reduction processing of reducing the body motion noise which is noise due to the body motion, on the basis of the body motion detection signal from the second light reception unit 142 and the body motion detection signal from the motion sensor unit 170. Then, frequency analysis processing such as FFT is performed with respect to the signal after being subjected to the body motion noise reduction processing, a spectrum is obtained, and processing is performed in which a representative frequency in the obtained spectrum is set to be a frequency of the heart rate. A value in which the obtained frequency increases 60 times is the number of pulses (the number of heart rates) which is generally used.

Furthermore, the beat information is not limited to the number of pulses, and for example, may be other various information items (for example, a frequency or a cycle of the heart rate, and the like) indicating the number of pulses. In addition, the beat information may be information indicating a beat state, and for example, a value indicating a blood volume may be the beat information.

The frequency analysis unit 212 performs the frequency analysis processing such as FFT with respect to the beat information, and thus a pulse spectrum is obtained. The pulse spectrum includes not only the frequency of the heart rate but also a frequency corresponding to a change (a fluctuation) in the frequency of the heart rate, and determines a sleep state by using the frequency.

The sleep state determination unit 216 determines the sleep state (for example, an REM sleep, a non-REM sleep, and the like) on the basis of the pulse spectrum. Specifically, a component of 0.04 Hz to 0.15 Hz in the pulse spectrum (hereinafter, referred to as an LF component) is an index indicating the activity of a sympathetic nerve of an automatic nerve and activity of a parasympathetic nerve, and a component of 0.15 Hz to 0.4 Hz (hereinafter, referred to as an HF component) is an index indicating the activity of the parasympathetic nerve. The LF component and the HF component are changed according to the sleep state, and thus the sleep state is determined by detecting the change. The determination of the sleep state is performed after initiation of sleep is determined and before an awakening is determined by the initiation of sleep and awakening determination unit 218 described later.

The initiation of the sleep and awakening determination unit 218 determines the initiation of sleep which proceeds to the sleep state from an awake state, and the awakening which proceeds to the awake state from the sleep state. As a determining method, various modification examples are considered, and for example, the user may perform a notification by pressing a button (the input unit 270) at bedtime, or the determination may be performed from the amount of the body motion which is detected by the motion sensor unit 170. Alternatively, the determination may be performed from a change in the body temperature which is detected by the temperature sensor unit 240.

The control unit 250 controls each unit of the biological information detecting device. Specifically, when the pulse wave is measured, intensity or a timing of the light emission of the light emission unit 150, a detection operation of the photoelectric sensor, a detection operation of the motion sensor unit 170, and the like are controlled. At this time, performing or stopping of the detection operation, and an intermittent operation are controlled according to a determination result of the sleep state, the initiation of sleep, or the awakening. For example, when it is determined as the non-REM sleep, a detection operation of the second light reception unit 142 which mainly detects the body motion noise is stopped.

The notification unit 260 (a notification device), for example, performs a notification of start-up at the time of turning an electric power source on, a notification of success of initial pulse wave detection, an alarm at the time of maintaining a state in which the pulse wave is not able to be detected for a constant period of time, a notification at the time of getting into the fat combustion zone, an alarm at the time of decreasing a battery voltage, a notification of a wake-up alarm, a notification of mail, a telephone call, or the like from a terminal device such as a smart phone, and the like. The notification unit 260, for example, is a light emission unit for a notification (an LED). Alternatively, the notification unit 260 may be a display unit such as an LCD or a buzzer, a vibration generation unit such as a vibration motor (a vibrator), and the like.

The input unit 270 receives an operation input from the user. For example, the input unit 270 is configured of a button and the like. As the operation input, for example, self-report of the initiation of sleep (bedtime) or the awakening (wake-up), the on and off of the electric power source, switchover of an operation mode, switchover of information to be displayed, starting and stopping of the pulse wave measurement, and the like are able to be assumed.

The communication unit 290 performs communication processing (reception processing, and transmission processing) with respect to the outside terminal device 420 as described in FIG. 1B. A function of the communication unit 290 is able to be realized by a processor for communication or a logic circuit such as an ASIC.

According to the embodiment described above, the biological information detecting device includes the first light reception unit 141 which receives light from the subject, the second light reception unit 142 which receives light from the subject, at least one light emission unit 150 which emits light to the subject, and the processing unit 200. The processing unit 200 determines an active state of the subject on the basis of the first detection signal detected by the first light reception unit 141 and the second detection signal detected by the second light reception unit 142, and controls a first detection operation of the light emission unit 150 and the first light reception unit 141 and a second detection operation of the light emission unit 150 and the second light reception unit 142 according to the active state. Here, as described later in FIG. 14A or the like, when a distance between the light emission unit 150 and the first light reception unit 141 is L1, and a distance between the light emission unit 150 and the second light reception unit 142 is L2, L1<L2 is satisfied.

As described later in FIGS. 14A and 14B and the like, the distance L1 between the light emission unit 150 and the first light reception unit 141 is less than the distance L2 between the light emission unit 150 and the second light reception unit 142 (L1<L2), and thus sensitivity with respect to the pulse wave and the body motion is different in the first light reception unit 141 and the second light reception unit 142. Accordingly, the detection signal of the pulse wave is mainly acquired by the first light reception unit 141, and the detection signal of the body motion is mainly acquired by the second light reception unit 142, and thus the body motion noise from the detection signal of the pulse wave is able to be reduced by the detection signal of the body motion.

At this time, the detection operation is controlled according to the active state, and thus it is possible to adaptively switchover the on and off of the detection operation (or the intermittent operation) according to the active state while effectively reducing the body motion noise by using two light reception units. Accordingly, it is possible to realize low power consumption even while using the two light reception units.

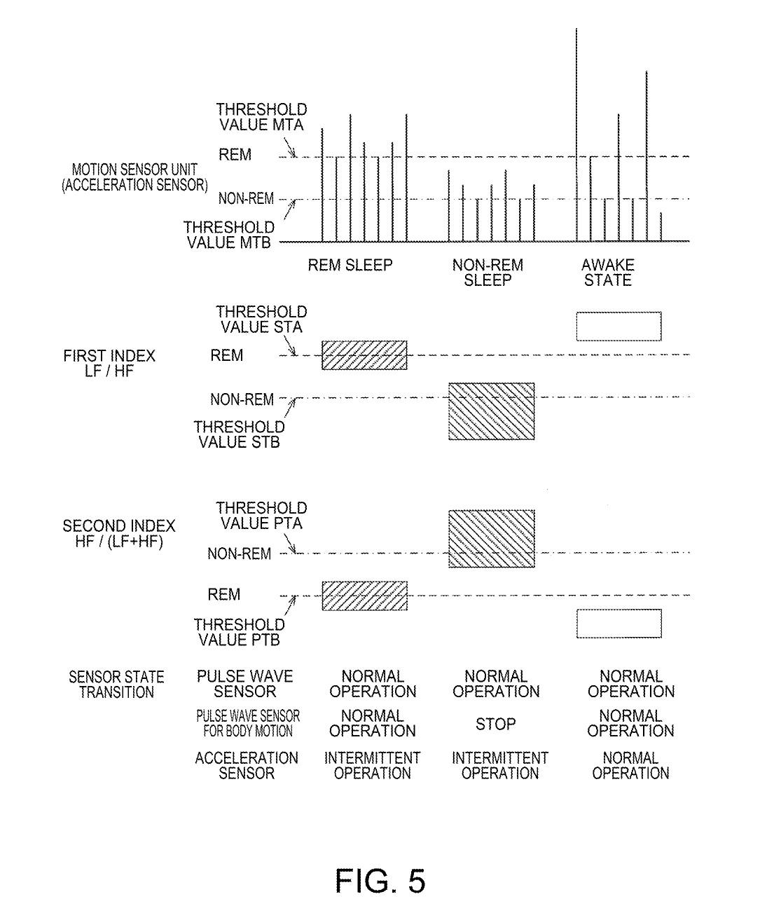

For example, in an example described later in FIG. 5, the active state is the REM sleep and the non-REM sleep into which the sleep state is classified. Then, the first detection operation which mainly detects the pulse wave is performed regardless of the sleep state, and the second detection operation which mainly detects the body motion is performed in the REM sleep and is stopped in the non-REM sleep. It is considered that this is because the non-REM sleep is a sleep state of a deep level, and thus the body motion is small, and even when the body motion noise is not reduced from the pulse wave detection signal, it is possible to detect the pulse wave with sufficient precision. Thus, it is possible to reduce power consumption of the second detection operation (and, the light emission of the light emission unit 150 at this time) in the non-REM sleep.

Furthermore, the active state is a state relevant to an activity level of the subject (the user), and for example, is the awake state and the sleep state. In this embodiment, a case where the awake state is one state and the sleep state is a plurality of states according to sleep stages will be described as an example, but the configuration is not limited thereto. For example, the awake state may be classified into a plurality of states according to the amount of the body motion, and the detection operation of the photoelectric sensor may be controlled according to the state. For example, a first state in which the body motion is comparatively small or less (for example, during desk work or the like), and a second state in which the body motion is comparatively large or great (for example, during movement or the like) are detected on the basis of the body motion detection signal obtained by the second light reception unit 142, and the detection operation may be set to an operation of low power consumption (for example, stopping a detection operation of a sensor for body motion) in the first state.

The sleep state is a state corresponding to a depth level of the sleep between the initiation of sleep and the awakening, and for example, is the REM sleep, and the non-REM sleep which is deeper than the REM sleep. In addition, the non-REM sleep is further classified into a shallow sleep, and a deep sleep which is deeper than the shallow sleep, the switchover of the on and off of the detection operation, and the intermittent operation may be controlled in the shallow sleep and the deep sleep. For example, when the sleep state is classified into six steps of the awakening, the REM sleep, and the REM sleep of level 1 to level 4, the non-REM sleep of level 1 may correspond to the shallow sleep, and the non-REM sleep of level 2 to 4 may correspond to the deep sleep.

In addition, in this embodiment, the active state is the sleep state of the subject. Then, when it is determined that the subject is in a first sleep state, the processing unit 200 sets the second detection operation to be in a normal operation mode, and when it is determined that the subject is in a second sleep state which is deeper than the first sleep state, the processing unit 200 sets the second detection operation to be in a non-operation mode.

In an example of FIG. 5 described later, the first sleep state is the REM sleep, and the second sleep state is the non-REM sleep. Furthermore, the configuration is not limited thereto, and the first sleep state and the second sleep state are able to be selected in various sleep states. For example, the REM sleep and the shallow sleep of the non-REM sleep may be set to the first sleep state, the deep sleep of the non-REM sleep may be set to the second sleep state, and the second detection operation may be set to be in the non-operation mode in the deep sleep of the non-REM sleep.

Accordingly, the detection operation is able to be more specifically controlled according to the depth level of sleep and not only the awakening and the sleep. That is, a sleep state in which the body motion comparatively easily occurs even in the sleep state is set to the first sleep state, and the detection operation of the second light reception unit 142 is performed in the first sleep state, and thus the body motion noise is able to be reduced. In contrast, a sleep state in which the body motion comparatively rarely occurs even in the sleep state and the body motion noise is rarely mixed into the pulse wave is set to the second sleep state, the detection operation of the second light reception unit 142 is stopped in the second sleep state, and thus low power consumption is able to be realized, and an available time (a time before it is necessary to charge a battery or replace a battery) is able to be extended. Transition between the sleep states fundamentally has a certain degree of length (for example, a few dozen minutes), and is repeated a plurality of times overnight, and thus a reduction in power consumption during the transition is extremely effective.

In addition, in this embodiment, when it is determined that the subject is in the awake state, the processing unit 200 sets the second detection operation to be in the normal operation mode, and when it is determined that the subject is in a predetermined sleep state, the processing unit 200 sets the second detection operation to be in the non-operation mode (an operation stop mode).

The predetermined sleep state is the non-REM sleep in the example of FIG. 5 described later, but the setting of the second detection operation to be in the non-operation mode is not limited to the non-REM sleep. For example, the second detection operation may be set to be in the non-operation mode in all of the sleep states (when it is determined as the initiation of sleep).

Accordingly, in the awake state where the amount of activity increases and the body motion noise is easily generated, the body motion noise is able to be reduced by using the second light reception unit 142, and high-precision pulse wave detection is able to be realized. Then, in the sleep state where the amount of activity decreases and the body motion noise is rarely generated, the detection operation is stopped by using the second light reception unit 142, and thus low power consumption is able to be realized, and an available time (a time before it is necessary to charge a battery or replace a battery) is able to be extended.

### 2. Determining Method of Sleep State

Hereinafter, the specification of the biological information detecting device described above will be described. First, a determining method of the sleep state will be described.

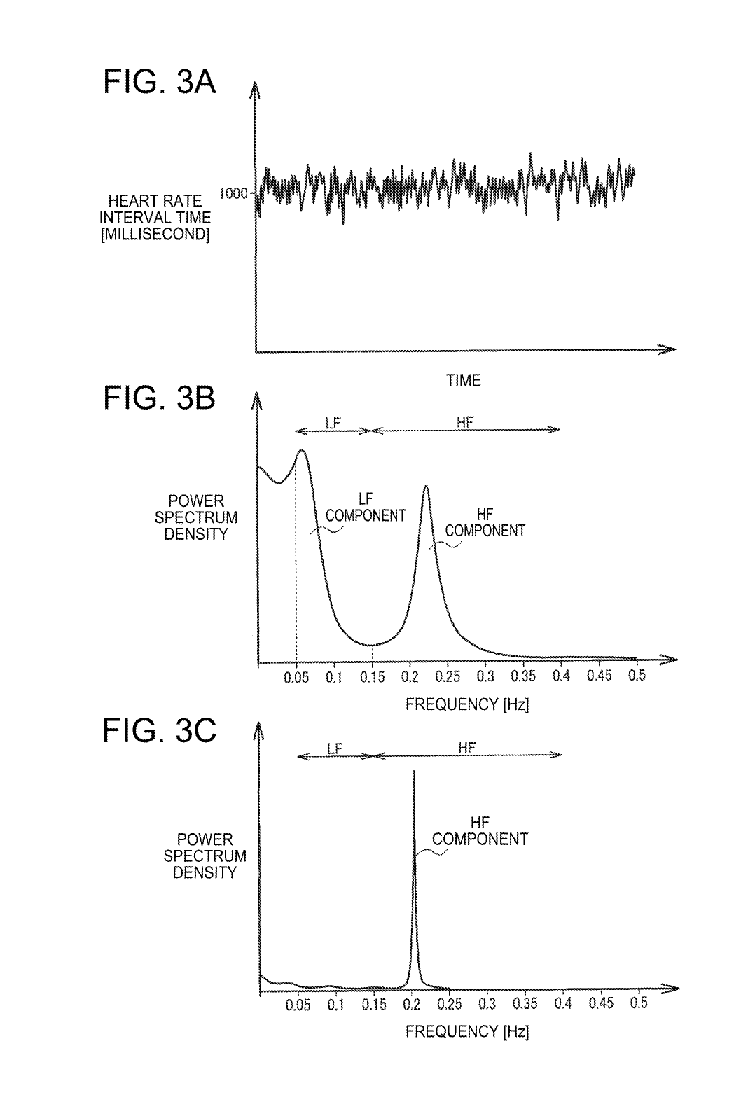

FIGS. 3A to 3C are diagrams illustrating the LF component and the HF component of the heart rate. FIG. 3A is an example of a temporal variation in a heart rate interval. The heart rate interval (a cycle) is a time from one beat to the next beat, and is approximately 1000 milliseconds in FIG. 3A. An inverse number thereof is the number of times of the beat (a frequency) per unit time, and thus corresponds to the beat of 1 time/second=60 times/minute. The heart rate interval fluctuates with a central focus on approximately 1000 milliseconds, and thus it is found that there is a temporal variation. In a fluctuation frequency, information indicating a state of the automatic nerve is included.

FIG. 3B is a power spectrum of the heart rate when the sympathetic nerve is superior to the parasympathetic nerve, and FIG. 3C is a power spectrum of the heart rate when the parasympathetic nerve is superior to the sympathetic nerve. As described above, the Low Frequency (LF) component corresponds to the component having a bandwidth of 0.04 Hz to 0.15 Hz, and the High Frequency (HF) component corresponds to the component having a bandwidth of 0.15 Hz to 0.4 Hz. For example, the component in each bandwidth is obtained by adding up (integrating) the power density in each bandwidth.

The sympathetic nerve is the automatic nerve which is easily activated when the subject performs brisk activity, and as illustrated in FIG. 3B, the LF component and the HF component appear together in the power spectrum of the heart rate. On the other hand, the parasympathetic nerve is the automatic nerve which is easily activated when the subject is resting, and as illustrated in FIG. 3C, approximately only the HF component appears in the power spectrum of the heart rate. Thus, the appearance and the size of the LF component and the HF component are changed according to a balance in a state of tension between the sympathetic nerve and the parasympathetic nerve, and thus, by using this, an activity balance in the automatic nerve is able to be estimated, and the sleep state is able to be determined according to the activity balance.

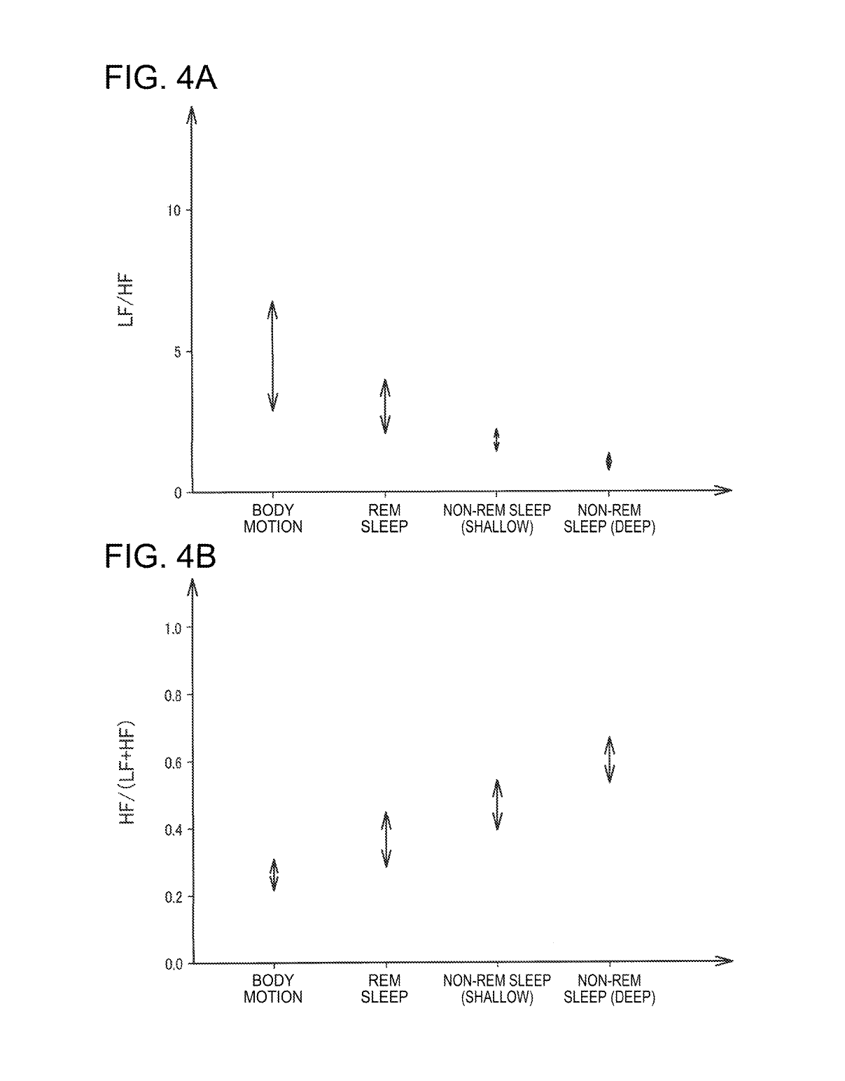

Specifically, a first index which is LF/HF and a second index which is HF/(LF+HF) are obtained from the LF component and the HF component, and these indexes are subjected to threshold value determination, and thus the sleep state is determined. An explanatory diagram thereof is illustrated in FIG. 4A to FIG. 5.

FIG. 4A is a diagram schematically illustrating a relationship between LF/HF and the active state. LF/HF is an index indicating activity of the sympathetic nerve, and indicates that the activity of the sympathetic nerve increases as a numerical value becomes greater. As illustrated in FIG. 4A, a value of LF/HF in each state has a width, and shows a trend in which LF/HF is maximized in the awake state (the body motion), and LF/HF decreases as the sleep becomes deeper.

In addition, FIG. 4B is a diagram schematically illustrating a relationship between HF/(LF+HF) and the active state. HF/(LF+HF) is an index indicating activity of the parasympathetic nerve, and indicates that the activity of the parasympathetic nerve increases as a numerical value becomes greater. As illustrated in FIG. 4B, a value of HF/(LF+HF) in each state has a width, and shows a trend in which HF/(LF+HF) is minimized in the awake state (the body motion), and HF/(LF+HF) increases as the sleep becomes deeper.

FIG. 5 is a diagram illustrating a determining method of the awake state and the sleep state, and control of the detection operation in each state. Furthermore, hereinafter, a case where the awake state is determined by LF/HF and HF/(LF+HF) will be described as an example, but the configuration is not limited thereto, and for example, only the sleep state may be determined by LF/HF and HF/(LF+HF), and the awake state may be determined by the other method.

In FIG. 5, a quadrangle illustrated in a portion of the first index and the second index schematically illustrates a distribution of index values in each state, and the value increases towards an upper direction in the sheet. LF/HF which is the first index is determined by a first threshold value STA and a second threshold value STB of LF/HF, and HF/(LF+HF) which is the second index is determined by a first threshold value PTA and a second threshold value PTB of HF/(LF+HF). Specifically, the determination is performed as follows.

STA<LF/HF, and HF/(LF+HF)<PTB: Awakening

STB<LF/HF<STA, and PTB<HF/(LF+HF)<PTA: REM sleep

LF/HF<STB, and PTA<HF/(LF+HF): Non-REM sleep

For example, the first threshold value STA of LF/HF is 5, and the second threshold value STB is 3. The first threshold value PTA of HF/(LF+HF) is 0.5, and the second threshold value PTB is 0.3. Furthermore, the value is an example, and a threshold value may be suitably set by an experiment or the like.

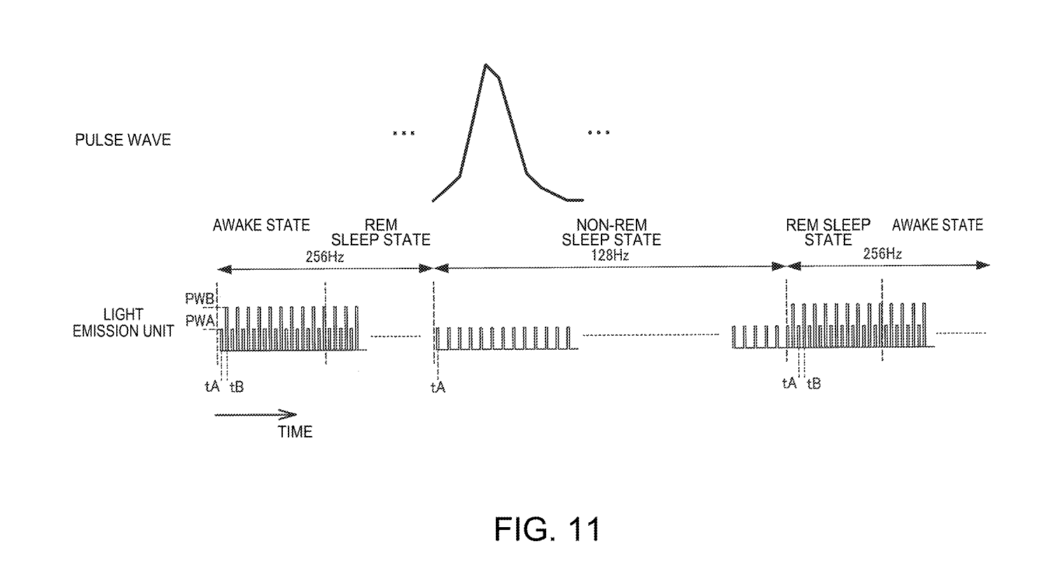

In the determination described above, when it is determined as the awake state or the REM sleep, both of the detection operations of the first light reception unit 141 (the pulse wave sensor) and the second light reception unit 142 (a pulse wave sensor for body motion) are set to a normal operation. In contrast, in the determination described above, when it is determined as the non-REM sleep, the detection operation of the first light reception unit 141 (the pulse wave sensor) is set to the normal operation, and the detection operation of the second light reception unit 142 (the pulse wave sensor for body motion) is stopped. A specific example of the normal operation or the stopping will be described later in FIG. 11 or the like.

In addition, in this embodiment, the awake state or the sleep state is further determined by using the acceleration sensor 172 (the motion sensor). In FIG. 5, a detection signal of the acceleration sensor 172 in each state is schematically illustrated by a vertical line. The time progresses in a rightward direction in the sheet, and an acceleration increases towards the upper direction in the sheet. The acceleration indicates the amount of the body motion, and the acceleration is changed by reflecting the amount of the body motion in each state, a variation in the amount of the body motion, or the like. For example, when a first threshold value of the acceleration is MTA, and a second threshold value is MTB (MTB<MTA), the number of times of MTA<Acceleration, the number of times of MTB<Acceleration<MTA, and the number of times of Acceleration<MTB within a predetermined period of time are counted. Then, the REM sleep, the non-REM sleep, and the awake state are determined by comparing these counted values.

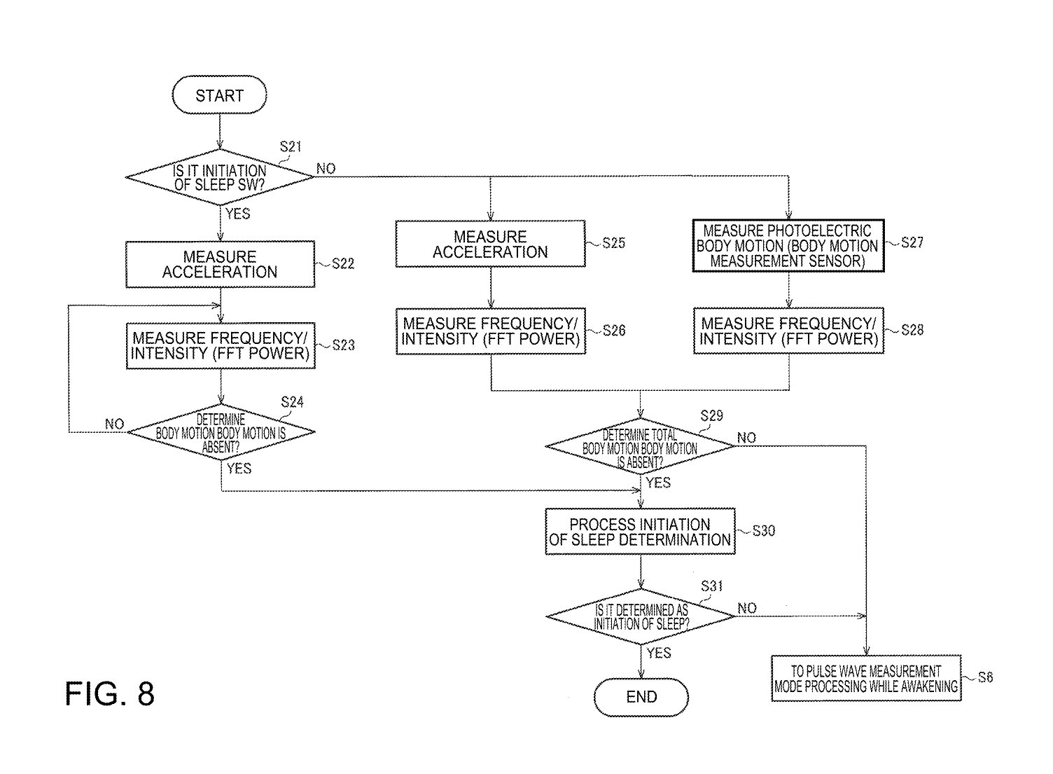

Alternatively, as described later in FIG. 8 or the like, the acceleration sensor 172 may be used for determining the initiation of sleep (or the awakening) regardless of the sleep state. In this case, the number of times that the acceleration exceeds a predetermined threshold value is detected, and it is determined whether or not the subject is in the awake state by substituting the number of times to a Cole equation.

The detection operation of the acceleration sensor 172 is also controlled according to the sleep state. That is, when it is determined as the awake state, the normal operation (constant detection) is performed, and when it is determined as the REM sleep or the non-REM sleep, the intermittent operation (intermittent detection) is performed.

Furthermore, the control of the detection operation according to the state transition is not limited to the configuration described above. For example, the detection operation of the second light reception unit 142 may not be stopped in the non-REM sleep, and may be in a low power consumption mode (for example, the intermittent operation). Alternatively, in the REM sleep or the non-REM sleep, the detection operation of the acceleration sensor 172 may be stopped without being in the intermittent operation.

### 3. Specification of Processing

Next, the specification of the determination processing of the sleep state and the control processing of the detection operation described above will be described.

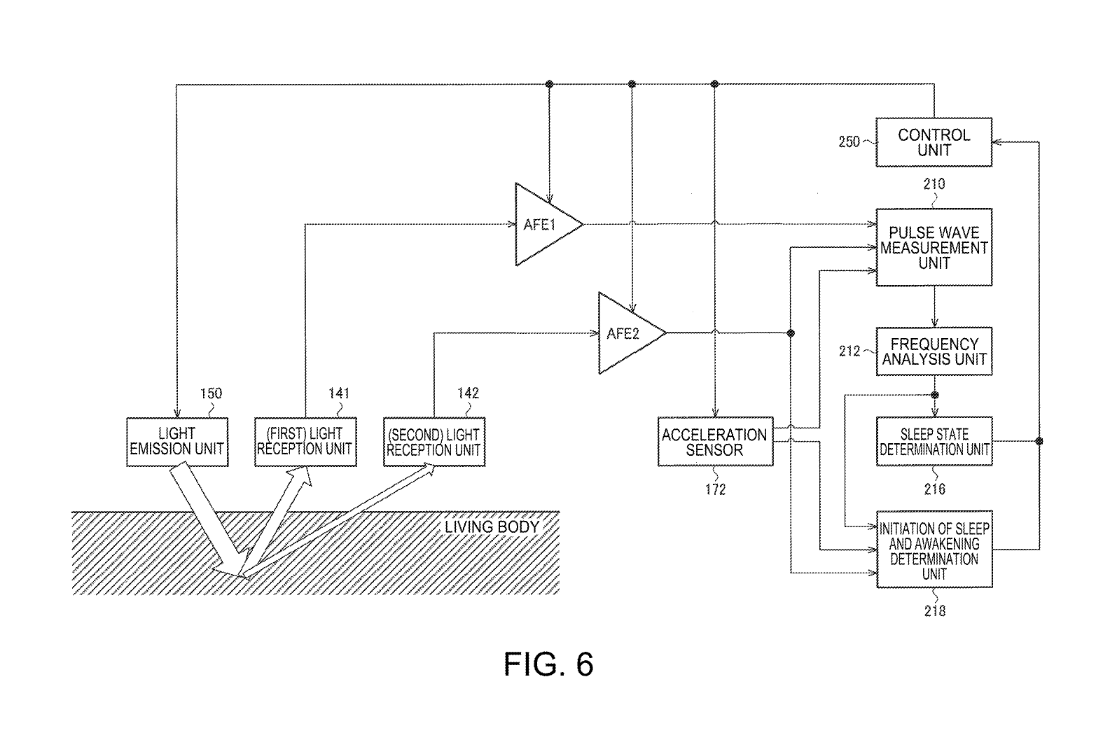

In FIG. 6, an example of a connection configuration of the biological information detecting device is illustrated. The biological information detecting device includes analog front end units AFE1 and AFE2, the first light reception unit 141, the second light reception unit 142, the light emission unit 150, the acceleration sensor 172, the pulse wave measurement unit 210, the frequency analysis unit 212, the sleep state determination unit 216, and the initiation of sleep and awakening determination unit 218. Furthermore, the same reference numerals are applied to the same constituents as those described above, and the description thereof will be suitably omitted.

The analog front end units AFE1 and AFE2, for example, are configured of an amplification circuit or a filter circuit, an A/D conversion circuit, and the like. The analog front end unit AFE1 performs amplification processing or filter processing with respect to the pulse wave detection signal from the first light reception unit 141, and performs A/D conversion with respect to the signal, and thus outputs the digital pulse wave detection signal to the pulse wave measurement unit 210. The analog front end unit AFE2 performs amplification processing or filter processing with respect to the body motion detection signal from the second light reception unit 142, performs A/D conversion with respect to the signal, and outputs the digital body motion detection signal to the pulse wave measurement unit 210. All or a part of the analog front end units AFE1 and AFE2, for example, may be embedded in the processing unit 200 (a CPU or the like), or may be disposed as a circuit element separately from the processing unit 200.

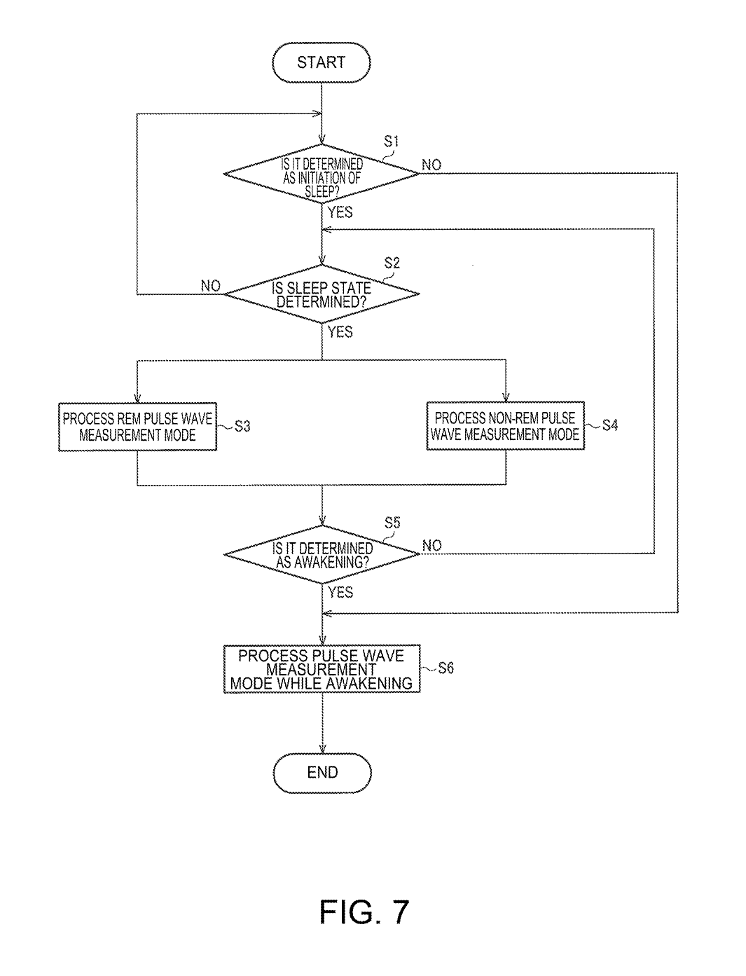

In FIG. 7, a flowchart of determining the active state and of controlling the detection operation is illustrated. When the processing is started, the initiation of sleep and awakening determination unit 218 determines whether or not it is the initiation of sleep on the basis of the body motion detection signal of the acceleration sensor 172 or the body motion detection signal of the second light reception unit 142 (Step S1).

When it is determined not to be the initiation of sleep, the control unit 250 and the pulse wave measurement unit 210 measure the pulse wave in the operation mode of the awakening (Step S6). In contrast, when it is determined as the initiation of sleep, the sleep state determination unit 216 determines the sleep state on the basis of the index value of LF/HF and HF/(LF+HF) (Step S2).

When it is determined as the awake state (that is, not the sleep state), the process returns to Step S1. In contrast, when it is determined as the REM sleep state, the control unit 250 and the pulse wave measurement unit 210 measure the pulse wave in the operation mode of the REM sleep (Step S3). In addition, when it is determined as the non-REM sleep state, the control unit 250 and the pulse wave measurement unit 210 measure the pulse wave in the operation mode of the non-REM sleep (Step S4).

Next, the initiation of sleep and awakening determination unit 218 determines whether or not it is the awakening on the basis of the index value of LF/HF and HF/(LF+HF) (Step S5). When it is determined not to be the awakening, the process returns to Step S2. In contrast, when it is determined as the awakening, the pulse wave is measured in the operation mode of the awakening (Step S6). Furthermore, here, the awakening determination is performed by the initiation of sleep and awakening determination unit 218, and the awakening determination may be performed by the sleep state determination unit 216 on the basis of the index value of LF/HF and HF/(LF+HF). In addition, the awakening determination is not limited to the configuration of using LF/HF and HF/(LF+HF), and for example, the awakening determination may be performed by using the acceleration sensor 172 or the like, similarly to the initiation of sleep determination.

### 4. Initiation of Sleep Determination Processing

Next, the specification of the processing in each step will be described.

In FIG. 8, a specific flowchart of the initiation of sleep determination processing in Step S1 is illustrated. When the processing is started, the initiation of sleep and awakening determination unit 218 determines whether or not a switch (the input unit 270) for performing the self-report of the initiation of sleep (the bedtime) is turned on by the user (Step S21).

When the switch is turned on, the body motion determination is performed by the acceleration. That is, the acceleration sensor 172 detects the acceleration signal (Step S22), and the initiation of sleep and awakening determination unit 218 performs the frequency analysis with respect to the acceleration signal (the FFT processing) and obtains a spectrum (Step S23). Then, the initiation of sleep and awakening determination unit 218 determines the presence or absence of the body motion from the spectrum (Step S24). For example, the presence or absence of the body motion is determined by whether or not the power which has a predetermined bandwidth (or, may have overall bandwidth) is greater than or equal to a threshold value. When it is determined that there is no body motion, the process proceeds to Step S30, and when it is determined that there is the body motion, the process returns to Step S23.

Incidentally, in Step S21 of FIG. 8, when the switch is not turned on, the body motion determination is performed by the acceleration and the detection signal of the photoelectric sensor. That is, the acceleration sensor 172 detects the acceleration signal (Step S25), and the initiation of sleep and awakening determination unit 218 performs the frequency analysis (the FFT processing) with respect to the acceleration signal and obtains a spectrum (Step S26). In addition, the initiation of sleep and awakening determination unit 218 acquires the body motion detection signal from the second light reception unit 142 (Step S27), performs the frequency analysis (the FFT processing) with respect to the body motion detection signal, and obtains a spectrum (Step S28). Then, the presence or absence of the body motion is determined from the spectrum of the acceleration signal and the spectrum of the body motion detection signal (Step S29).

A method of determining the presence or absence of the body motion from the spectrum of the acceleration signal is identical to that described above. A method of determining the presence or absence of the body motion from the spectrum of the body motion detection signal, for example, similarly to a case of the acceleration signal, determines the presence or absence of the body motion by whether or not the power which has a predetermined bandwidth (or, may have overall bandwidth) is greater than or equal to a threshold value.

When it is determined that there is no body motion, the process proceeds to Step S30, and when it is determined that there is the body motion, it is determines as the awake state, and the process proceeds to Step S6.

In Step S30, the initiation of sleep and awakening determination unit 218 performs the initiation of sleep determination on the basis of the detection signal of the acceleration sensor 172. For example, the initiation of sleep determination is performed by a Cole equation. In the determination performed by using the Cole equation, the before and after of a time point of the determination is divided into a plurality of periods, and the number of times that the acceleration exceeds a predetermined threshold value in each period is counted. The Cole equation is an equation of weighting and adding the counted value in each period, a value of the Cole equation is obtained by substituting the counted value to the Cole equation, and the value is subjected to the threshold value determination.

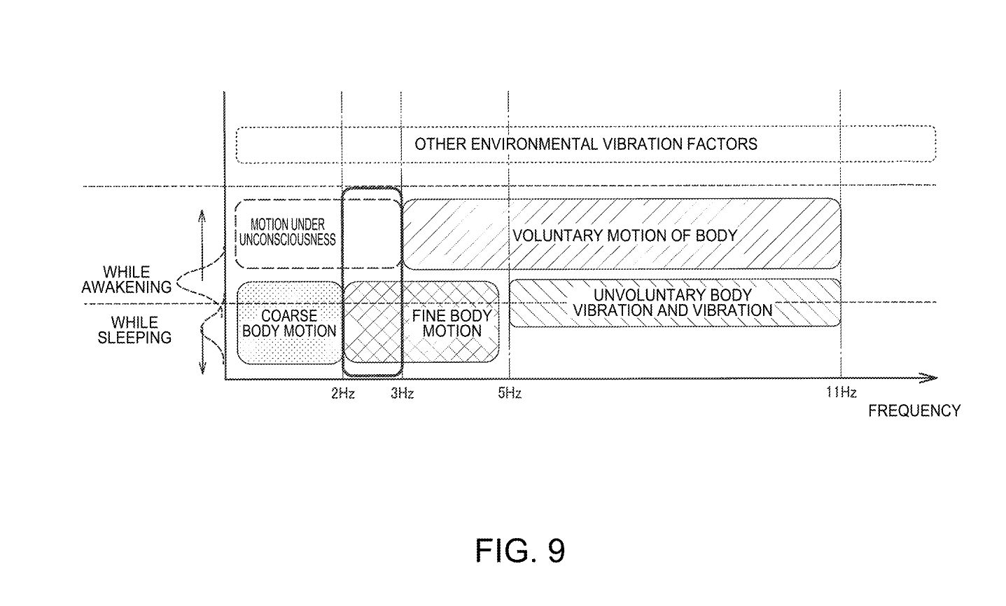

The frequency of the body motion will be described with reference to FIG. 9 by using the initiation of sleep determination. Furthermore, the frequency of the body motion described later may be used not only in the initiation of sleep determination, but also in the body motion determination such as Steps S24 and S29.

FIG. 9 is a diagram illustrating vibration factors. While awakening, unconscious body motion or voluntary motion, coarse body motion, fine body motion, and involuntary vibration are generated, and while sleeping, the coarse body motion, the fine body motion, and the involuntary vibration are generated. In addition, there is vibration generated from the environment regardless of the awakening or the sleep. A vibration frequency of each factor is illustrated in the drawing.

Among the vibration factors, the unconscious body motion is adopted as a vibration factor suitable for the initiation of sleep determination. The reason of this is as follows. That is, the coarse body motion is the body motion or contraction of a plurality of muscles which is sustained for greater than or equal to 0.5 seconds, and the fine body motion is the body motion or contraction of a single muscle which is sustained for less than 0.5 seconds. From this, it is considered that the fine body motion occurs for a short sustained period or has small movements (the acceleration), and it is considered that the fine body motion rarely becomes a barrier at the time of detecting the other body motion. The unconscious body motion and the voluntary motion are able to be generated in the bandwidth of the fine body motion, the voluntary motion is not necessarily constantly generated, and thus it is considered that it is suitable to distinguish the sleep from the awakening in the unconscious body motion.

The bandwidth of the unconscious body motion and the bandwidth of the fine body motion overlap each other in a bandwidth of 2 Hz to 3 Hz, and a signal of the bandwidth is obtained from the acceleration signal by bandpass filter processing. Then, it is determined whether or not the signal of the bandwidth of 2 Hz to 3 Hz is greater than or equal to a threshold value, and when the signal is greater than or equal to the threshold value, the counted value is incremented.

When the value of the Cole equation obtained in Step S30 is greater than or equal to the threshold value, it is determined as the initiation of sleep, and the processing ends. In contrast, when the value of the Cole equation is less than the threshold value, it is determined as the awake state, and the process proceeds to Step S6.

Furthermore, the initiation of sleep determination is not limited to the method described above, and various modification examples are considered. For example, the initiation of sleep determination may be performed on the basis of time (detection of a predetermined time) or body temperature (detection of a fluctuation in body temperature), a detection signal of a gyro sensor, a detection signal of an Inertial Measurement Unit (IMU), a detection signal of a GPS (detection of a movement amount), atmospheric pressure (detection of an atmospheric pressure difference between an upright position and a recumbent position), breathing, and the like.

### 5. Sleep State Determination Processing

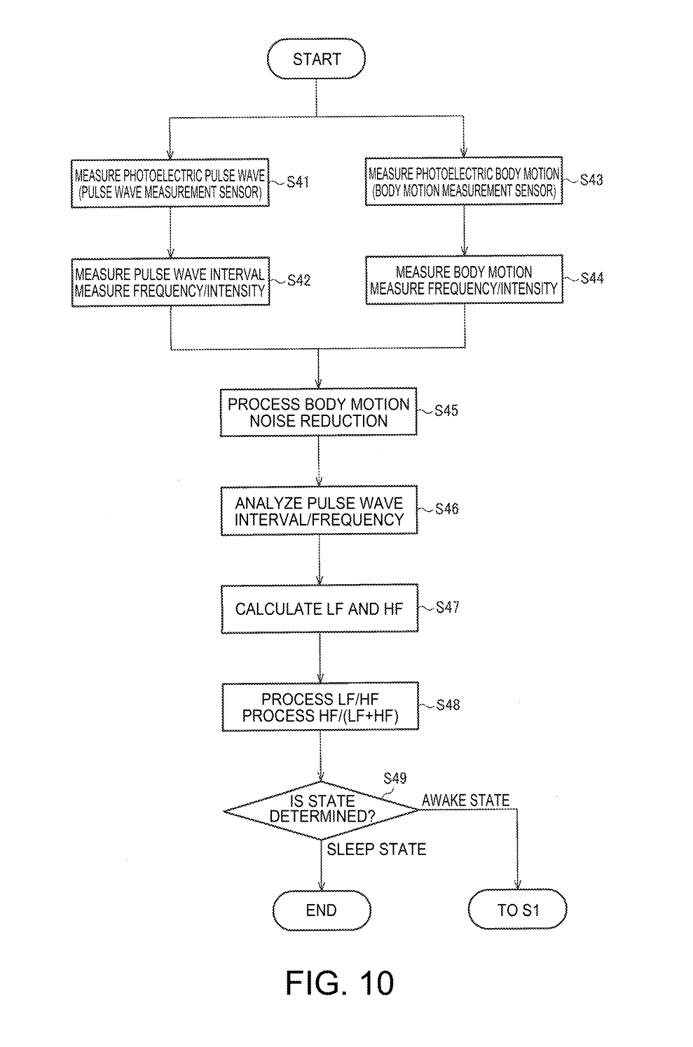

In FIG. 10, a specific flowchart of the sleep state determination processing of Step S2 is illustrated. When the processing is started, the control unit 250 allow the light emission unit 150 to emit light, and the pulse wave measurement unit 210 acquires the pulse wave detection signal from the first light reception unit 141 and the body motion detection signal from the second light reception unit 142 (Steps S41 and S43).

Next, the pulse wave measurement unit 210 performs the frequency analysis (the FFT processing) with respect to the pulse wave detection signal and the body motion detection signal, and obtains the spectrum (Steps S42 and S44), and processing of reducing the body motion noise from the pulse wave detection signal is performed by the spectral subtraction (Step S45). Furthermore, adaptive filter processing using the signal from the motion sensor unit 170 may be further performed, and the body motion noise may be reduced.

Next, the frequency analysis unit 212 performs the frequency analysis (the FFT processing) with respect to the pulse wave detection signal, and obtains the spectrum of the pulse wave (Step S46). Next, the frequency analysis unit 212 obtains the LF component and the HF component from the spectrum of the pulse wave (Step S47), and obtains the first index LF/HF and the second index HF/(LF+HF) (Step S48).

Next, the sleep state determination unit 216 determines the sleep state on the basis of the first index LF/HF and the second index HF/(LF+HF) (Step S49). A determining method is as described in FIG. 3A to FIG. 5. When it is determined as the awake state, the process returns to Step S1, and when it is determined as the REM sleep or the non-REM sleep, the sleep state determination processing ends.

### 6. Detection Operation of Photoelectric Sensor

Next, the detection operation in the operation mode of the REM sleep in Step S3, the operation mode of the non-REM sleep in Step S4, and the operation mode of the awakening in Step S6 will be described.

FIG. 11 is a timing chart of a light emission operation in each operation mode. The waveform of the pulse wave schematically illustrates an intensity of reflected light which is incident on the light reception unit in the beat. In the waveform of the light emission unit, each pulse indicates a light emitting timing, and the height of the pulse indicates a light intensity (light emitting power).

In the awake state and the REM sleep state, the light emission unit 150 emits light at a timing to and a first light intensity PWA, and emits light at a timing tB and a second light intensity PWB. The second light intensity PWB is greater than the first light intensity PWA. The pulse wave detection signal is acquired by the first light reception unit 141 at the timing tA, and the body motion detection signal is acquired by the second light reception unit 142 at the timing tB. The timing tA and the timing tB, for example, are alternate, and a frequency of the light emission including both of the timings, for example, is 256 Hz.

In the non-REM sleep state, the acquisition of the body motion detection signal of the second light reception unit 142 is stopped. That is, the light emission unit 150 emits light at the timing tA and the first light intensity PWA, and does not emit light at the timing tB. The pulse wave detection signal is acquired by the first light reception unit 141 at the timing tA. The frequency of the light emission at the timing tA, for example, is 128 Hz. The frequency (the number of times) of the light emission becomes ½, and the light emission of which the light intensity is greater is not performed, and thus power consumption in the light emission unit 150 is suppressed.

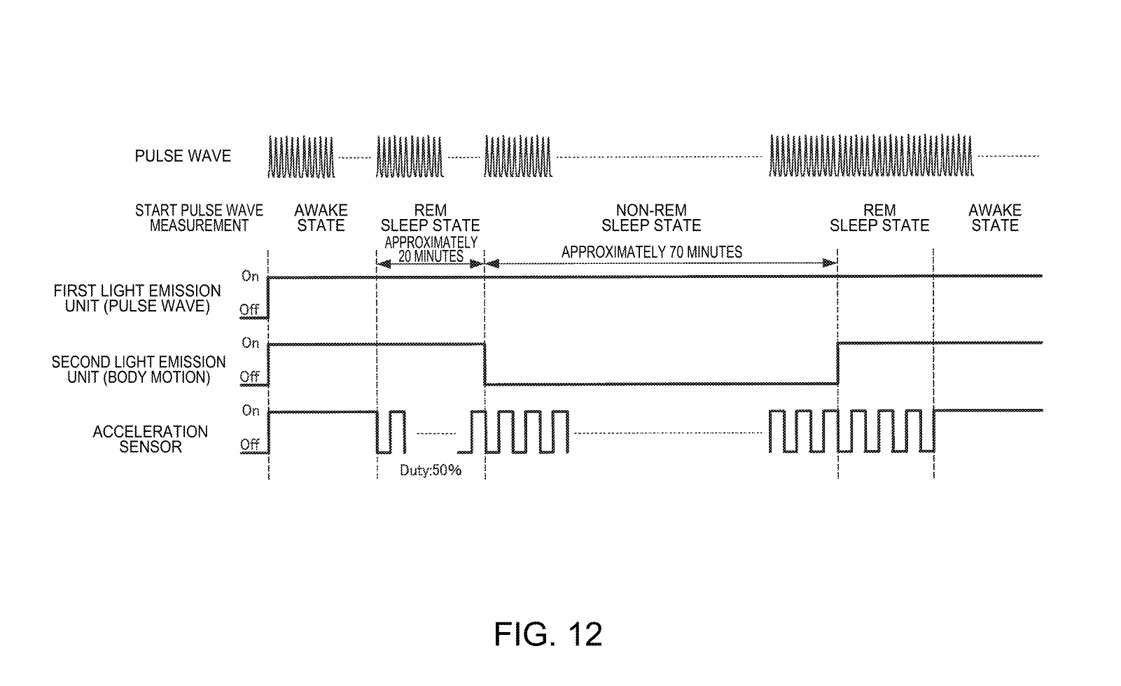

FIG. 12 is an operation timing chart of the photoelectric sensor and the acceleration sensor in each operation mode. As a general duration time of the REM sleep and the non-REM sleep, 20 minutes and 70 minutes are described, and in an actual operation, the duration time may be an arbitrary duration time.

The detection operation of the first light reception unit 141 is performed in all of the awake state, the REM sleep state, and the non-REM sleep state. In a detection operation “on” period, the detection signal is acquired at the timing tA described in FIG. 11. The timing tA, for example, is a timing that the A/D conversion circuit of the analog front end unit AFE1 obtains an analog detection signal.

The detection operation of the second light reception unit 142 is performed in the awake state and the REM sleep state, and is not performed in the non-REM sleep state. In the detection operation “on” period, the detection signal is acquired at the timing tB described in FIG. 11. The timing tB, for example, is a timing that the A/D conversion circuit of the analog front end unit AFE2 obtains an analog detection signal. In a detection operation “off” period, the control unit 250 sets the analog front end unit AFE2 to be in the operation stop mode. For example, a switch element (for example, a transistor or the like) is disposed in an electric power source supply line of the analog front end unit AFE2, and the control unit 250 turns the switch element off, and thus the operation is stopped. Alternatively, only the amplification circuit among the amplification circuit and the A/D conversion unit may turn the supply of the electric power source off. Alternatively, a bias current (or a bias voltage) of the analog front end unit AFE2 may be turned off, and thus the operation may be stopped.

The detection operation of the acceleration sensor 172 is continuously performed in the awake state, and is intermittently performed in the REM sleep state and the non-REM sleep state. The length of the operation “on” period in the intermittent operation, for example, is set to a length required for calculation of the FFT processing or the Cole equation. The duty between the operation “on” and the operation “off” in the intermittent operation, for example, is 50%, but the configuration is not limited thereto.

As described above, the detection signal of the second light reception unit 142 and the detection signal of the acceleration sensor 172 are fundamentally used for reducing the body motion noise, and in this embodiment, the signal used for reducing the body motion noise is changed according to the sleep state.

That is, the body motion increases in the awake state, and thus the detection signal of the second light reception unit 142 and the detection signal of the acceleration sensor 172 are used together in order to reduce the body motion noise with high precision. For this reason, both of the detection operations are turned “on”. The body motion decreases in the REM sleep state, and thus only the detection signal of the second light reception unit 142 is used, but the detection signal of the acceleration sensor 172 is not used. For this reason, the detection operation of the acceleration sensor 172 becomes the intermittent operation, and thus power consumption is reduced. In the non-REM sleep state, the body motion rarely occurs, and it is not necessary to reduce the body motion noise, and thus the detection signal of the second light reception unit 142 and the detection signal of the acceleration sensor 172 are not used together. For this reason, the detection operation of the second light reception unit 142 is turned “off”, and the detection operation of the acceleration sensor 172 becomes the intermittent operation, and thus power consumption is reduced.

As described above, the detection signal of the acceleration sensor 172 is not used for reducing the body motion noise in the REM sleep state and the non-REM sleep state, but is used as subsidiary data for increasing accuracy of detecting the transition in the sleep states. For this reason, the detection operation of the acceleration sensor 172 becomes the intermittent operation without constantly performing the measurement. For example, as described in FIG. 5, the sleep state may be determined by the threshold value determination of the acceleration, and a determination result thereof may be used as subsidiary of the determination of LF/HF or the like.

### 7. Awakening Determination Processing

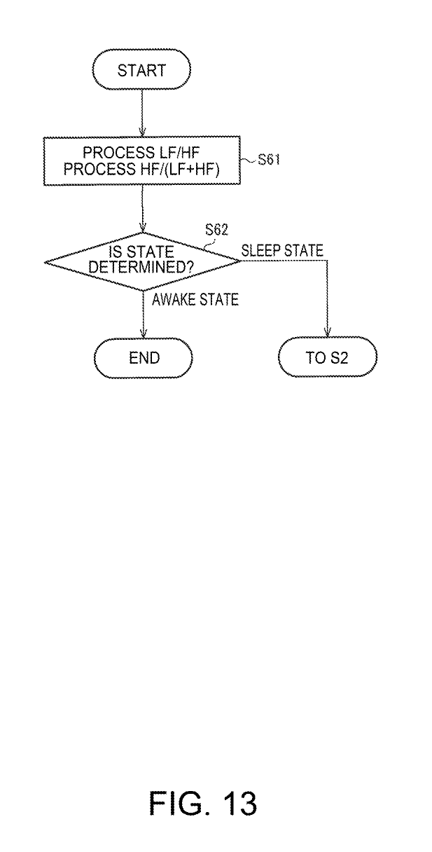

In FIG. 13, a specific flowchart of the awakening determination processing in Step S5 is illustrated. When the processing is started, the frequency analysis unit 212 obtains the first index LF/HF and the second index HF/(LF+HF) (Step S61). Next, the initiation of sleep and awakening determination unit 218 determines whether or not it is the awake state on the basis of the first index LF/HF and the second index HF/(LF+HF) (Step S62). A determining method is as described in FIG. 3A to FIG. 5. When it is determined as the sleep state, the process returns to Step S2, and when it is determined as the awake state, the awakening determination processing ends.

According to the embodiment described above, the processing unit 200 allows the light emission unit 150 to emit light during a first period of detecting the light from the subject by the first light reception unit 141 and a second period of detecting the light from the subject by the second light reception unit 142 in the normal operation mode of the second detection operation. In contrast, the processing unit 200 stops the light emission of the light emission unit 150 during the second period in the non-operation mode of the second detection operation in a predetermined sleep state.

For example, in examples of FIG. 11 and FIG. 12, the second detection operation of the second light reception unit 142 is in the normal operation mode at the awakening and the REM sleep, and is in the non-operation mode (stop) at the non-REM sleep. Then, the light emission unit 150 emits light during the first period (the timing tA) and the second period (timing tB) at the awakening and the REM sleep, and the light emission unit 150 does not emit light during the second period (the timing tB) at the non-REM sleep.

The non-REM sleep of the sleep state is deep sleep, and the body motion is small in the non-REM sleep (for example, the size of one body motion is small, or the frequency of the body motion is low). For this reason, the second detection operation of the second light reception unit 142 which mainly detects the body motion noise is able to be in the non-operation mode. As described above, the non-operation mode, for example, corresponds to the fact that the operation of the analog front end unit AFE2 is stopped. Further, the light emission in the second period is not necessary, and thus the light emission in the second period is able to be stopped in the non-operation mode. Thus, in the sleep state where the body motion is small, power consumption in the light emission unit 150 or the analog front end unit AFE2 is able to be reduced, and thus an available time of a battery in a portable device is able to be extended. In the example of FIG. 11, the non-REM sleep is for approximately 70 minutes, and is repeated a plurality of times overnight. During this time, the light emission operation which continuously blinks is able to be stopped, and thus the number of times of the light emission is considerably reduced, and a reduction effect in power consumption increases.

In addition, in this embodiment, the processing unit 200 performs the body motion noise reduction processing which reduces the body motion noise of the first detection signal of the first light reception unit 141 on the basis of the second detection signal of the second light reception unit 142. Then, the processing unit 200 calculates the biological information on the basis of the first detection signal after being subjected to the body motion noise reduction processing.

It is possible to make sensitivity with respect to the pulse wave and the body motion different in each of the light reception units by including the first light reception unit 141 and the second light reception unit 142. For example, it is possible to change the sensitivity with respect to the biological information and the body motion by making the distance (L1 and L2 in FIG. 14A) between the light emission unit 150 and each of the light reception units, or the height (h1 and h2 in FIG. 14A) of the light transmissive member 50 in each of the light reception units different. Accordingly, it is possible to reduce the body motion noise from the first detection signal which mainly detects the biological information but includes the body motion noise mixed therein, by using the second detection signal which mainly detects the body motion noise. Thus, the body motion noise is reduced, and thus the biological information with high precision (for example, an S/N ratio is high) is able to be calculated.

In addition, in this embodiment, the processing unit 200 obtains the pulse wave information as the biological information, and determines the active state on the basis of the pulse wave information.

As described in FIGS. 3A to 3C and the like, the pulse wave information is associated with the activity balance of the automatic nerve, and the activity balance of the automatic nerve is changed according to the active state (the awakening, the REM sleep, and the non-REM sleep). By using this, it is possible to determine the active state from the pulse wave information. For example, in an example of FIGS. 3A to 3C, the pulse wave information is a beat interval (the number of pulses), and a fluctuation in the beat interval is changed according to the activity balance of the automatic nerve. The fluctuation in the beat interval is detected, and the active state is able to be determined with reference to a determination criterion in each active state.

Specifically, the processing unit 200 obtains the first index LF/HF and the second index HF/(LF+HF) by the frequency analysis of the pulse wave information, and determines the active state on the basis of the first index LF/HF and the second index HF/(LF+HF). As described in FIG. 4A and FIG. 4B, the first index LF/HF indicates the activity of the sympathetic nerve, and the second index HF/(LF+HF) indicates the activity of the parasympathetic nerve.

As described in FIG. 3B or the like, the LF component and the HF component are information of the fluctuation of the beat interval. It is possible to obtain information indicating the activity balance of the automatic nerve from the pulse wave information by obtaining the first index LF/HF and the second index HF/(LF+HF) from the LF component and the HF component. Then, it is possible to estimate the active state from the activity balance of the automatic nerve by using the first index LF/HF and the second index HF/(LF+HF).

In addition, in this embodiment, the motion sensor unit 170 detects the body motion information of the subject, and the processing unit 200 determines the active state of the subject on the basis of the body motion information. Then, when it is determined that the subject has transitioned from the awake state to the sleep state (the initiation of sleep), the processing unit 200 sets the motion sensor unit 170 to be in the low power consumption mode.

For example, as described in FIG. 8 or the like, the motion sensor unit 170 detects the acceleration signal as the body motion information, and the processing unit 200 determines whether or not the subject is in the initiation of sleep by obtaining the Cole equation from the acceleration signal having a bandwidth of 2 Hz to 3 Hz. When it is determined as the initiation of sleep, the processing unit 200 sets the operation mode at the REM sleep or the operation mode at the non-REM sleep. As described in FIG. 12 or the like, in these operation modes, the motion sensor unit 170 is set to be in the low power consumption mode (for example, the intermittent operation).

As described above, the body motion information detected by the motion sensor unit 170 is used for the body motion noise reduction processing, and the body motion decreases after the initiation of sleep, and thus it is possible to calculate the biological information with sufficient precision (for example, S/N) without reducing the body motion noise by the motion sensor unit 170. For this reason, it is possible to suppress power consumption in the motion sensor unit 170 by setting the motion sensor unit 170 to be in the low power consumption mode. In the example of FIG. 12, the duty is 50%, and thus power consumption becomes approximately ½. On the other hand, it is possible to intermittently obtain the acceleration signal by setting the motion sensor unit 170 to be in the low power consumption mode without completely stopping the motion sensor unit 170. For example, as described in FIG. 5, it is possible to more accurately estimate the sleep state with the pulse wave by using the acceleration signal for determining the sleep state.

### 8. Sensor Unit

Next, the arrangement of the photoelectric sensor and a relationship between the shape of the light transmissive member 50 and the pulse wave detection will be described. First, in FIGS. 14A and 14B, an example of a specific configuration of the sensor unit 40 is illustrated.

FIG. 14A is a sectional view of the sensor unit 40, and FIG. 14B is a plan view illustrating the arrangement of the light emission unit 150, the first light reception unit 141, and the second light reception unit 142 on the substrate 160. FIG. 14B corresponds to a planar view at the time of performing observation in a direction (a direction of DR2) from the subject side to the biological information detecting device in a mounting state of FIG. 14A.

The first light reception unit 141, the second light reception unit 142, and the light emission unit 150 are mounted on the substrate 160 (a sensor substrate). The light emission unit 150 emits the light to the subject, the light is reflected or transmitted by the subject (for example, a blood vessel or the like), and the first light reception unit 141 and the second light reception unit 142 receive and detect the reflected light or transmitted light. The first light reception unit 141 and the second light reception unit 142, for example, are able to be realized by a light reception element such as a photodiode. An angle limiting filter of narrowing a light reception angle or a wavelength limiting filter of limiting the wavelength of the light which is incident on the light reception element may be formed on a diode element. The light emission unit 150, for example, is able to be realized by a light emission element such as an LED. Furthermore, it is not necessary that all of the first light reception unit 141, the second light reception unit 142, and the light emission unit 150 are mounted on the same substrate 160, and at least a part of these elements (for example, the second light reception unit 142) may be separately disposed on the substrate.

In a case of a pulsimeter, the light from the light emission unit 150 advances into the subject, and is diffused or scattered in a surface skin, an inner skin, a subcutaneous tissue, and the like. After that, the light reaches the blood vessel (a portion to be detected), and is reflected. At this time, a part of the light is absorbed by the blood vessel. Then, an absorption rate of the light in the blood vessel is changed due to an influence of the pulse, a light intensity of the reflected light is also changed, and thus the first light reception unit 141 receives the reflected light, and the number of pulses or the like which is the biological information is able to be detected by detecting a change in the light intensity.

Furthermore, a light shielding wall 70 (a member for light shielding) which shields direct light from the light emission unit 150 to the first light reception unit 141 and the second light reception unit 142 may be disposed between the first light reception unit 141 and the light emission unit 150.

The light transmissive member 50 is disposed on a surface of the biological information detecting device which is in contact with the subject, and transmits the light from the subject. In addition, the light transmissive member 50 is in contact with the subject at the time of measuring the biological information of the subject. For example, the convex portion 52 is formed in the light transmissive member 50, and the convex portion 52 is in contact with the subject. It is preferable that the surface of the convex portion 52 is in the shape of a curved surface (a spherical surface), but the configuration is not limited thereto, and various shapes are able to be adopted. In addition, the light transmissive member 50 may be transparent with respect to the wavelength of the light from the subject, and a transparent material may be used or a chromatic material may be used.

In this embodiment, a plurality of photoelectric sensors is realized by disposing a plurality of light reception units, and thus a plurality of convex portions 52 (for example, the number of convex portions corresponds to the number of photoelectric sensors) may be disposed. In an example of FIG. 14A, a convex portion 52-1 is disposed in the first photoelectric sensor which is realized by the light emission unit 150 and the first light reception unit 141, and a convex portion 52-2 is disposed in the second photoelectric sensor which is realized by the light emission unit 150 and the second light reception unit 142.

Furthermore, not only the light transmissive member 50 but also a contact portion 80 which stabilizes a contact state between the sensor unit 40 and the subject may be disposed. The contact portion herein, for example, is “80” in FIG. 14A, and as an example, is disposed around the light emission unit 150, the first light reception unit 141, and the second light reception unit 142. When such a contact portion 80 is disposed, it is assumed that the biological information detecting device is fixed to the subject in a state where a pressure is (idealistically) equally applied in the contact portion 80. That is, a flat surface defined by the contact portion 80 is a surface indicating a criterion in the mounting of the biological information detecting device. In this case, it is possible to make a difference in a pressing force between a position (h1) higher than the surface which is the criterion and a position (h2) lower than the surface definitive.

Next, the arrangement of the light reception unit and the height of the light transmissive member 50 will be described. As illustrated in FIGS. 14A and 14B, the light emission unit 150, the first light reception unit 141, and the second light reception unit 142 are arranged along a predetermined direction of the substrate 160 (a rightward direction in the sheet). The distance L2 between the second light reception unit 142 and the light emission unit 150 is greater than the distance L1 between the first light reception unit 141 and the light emission unit 150 (L2>L2). Here, the distances L1 and L2, for example, is a distance between the light emission unit 150 and a representative position of each of the light reception units and a distance along the predetermined direction of the substrate 160. The representative position of the light reception unit, for example, may be a center position of the light reception unit indicated by A1 and A2 (for example, the center of the light reception region in the photodiode or the like). When a lens is disposed in the light emission unit 150, the center position of the light emission unit 150, for example, is the center of the lens, the center of the light emit region in the light emit diode, and the like.

The direction of the height of the light transmissive member 50 is a direction (DR1 of FIG. 14A) which is directed towards the subject from the biological information detecting device in a state where the biological information detecting device is mounted. The height h1 of the light transmissive member in a position and a region corresponding to the first light reception unit 141 is higher than the height h2 of the light transmissive member in a position or a region corresponding to the second light reception unit 142 (h1>h2).

A defining method of the height is able to be variously modified, and for example, as illustrated in FIG. 14A, a distance between the substrate 160 (a surface of the substrate 160 on which the light emission unit 150 or the like is disposed) and a surface which is in contact with the subject of the light transmissive member 50 in a region where the light transmissive member 50, the first light reception unit 141, and the second light reception unit 142 overlap each other may be the height in a plan view of the direction of DR2. The distance may be the distance (the height) in the representative position as described above, or may be an average distance (an average height) in the region. Alternatively, the thickness itself of the light transmissive member 50 may be the height. Alternatively, a criterion surface which is parallel with the surface of the substrate 160 (for example, an imaginary surface, and a surface of any member) may be set, and a distance from the criterion surface may be the height of the light transmissive member 50.

In addition, a defining method of the position or the region corresponding to each of the light reception units is also variously considered. For example, each of the heights h1 and h2 is a height of the light transmissive member 50 in a representative position of the first light reception unit 141 and the second light reception unit 142. Here, as the representative position, for example, the center positions A1 and A2 of the respective light reception units or the like may used. For example, by defining an intersection point between a straight line extending from A1 to the direction of DR1 and the surface of the light transmissive member 50 (a surface which is in contact with the subject at the time of mounting the light transmissive member 50), the height h1 of the light transmissive member 50 in the intersection point may be used as the height of the light transmissive member 50 in the center position A1. Alternatively, the height h1 may be an average height of the light transmissive member 50 in a region where the light transmissive member 50 and the first light reception unit 141 overlap each other (or including the first light reception unit 141) in a plan view from the subject side to the direction of DR2. A region where the light transmissive member 50 and the light reception unit overlap each other (or including the light reception unit) is also variously considered, and for example, a region which is coincident with a light reception region of the photodiode forming the first light reception unit 141, or a region which includes the light reception region and has the minimum area (for example, in the shape of a rectangle) in a plan view of the direction of DR2 may be considered.

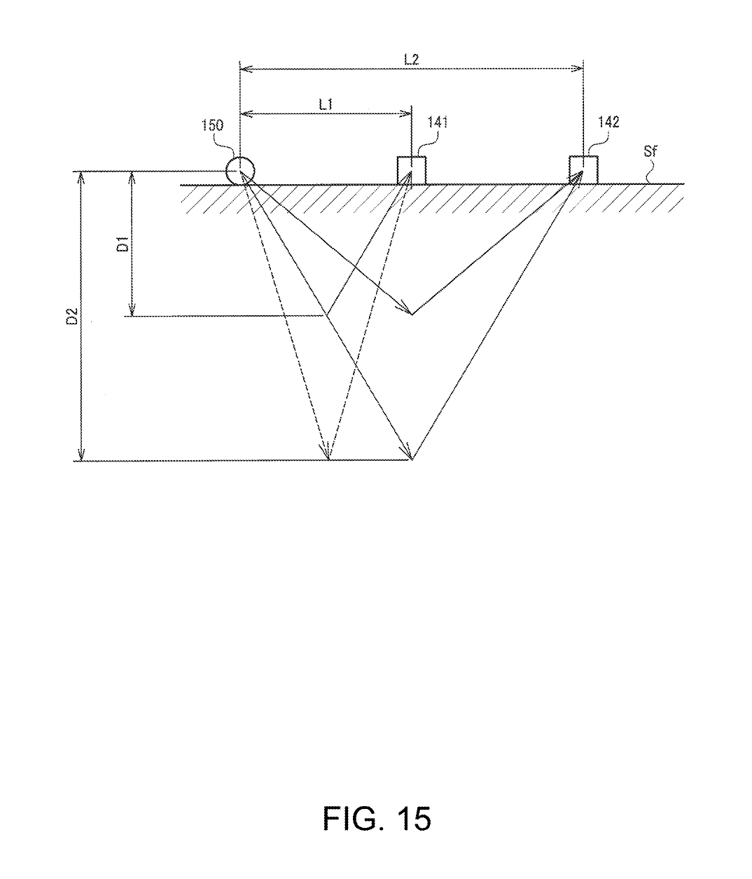

As described later in FIG. 15 or the like, when the distance between the light emission unit 150 and the light reception unit is different, a route in which light passes through the tissue or a depth in which the light reaches the tissue is changed. A light intensity which reaches the light reception unit increases and detection sensitivity of the signal increases as the distance from the light emission unit 150 becomes shorter, and thus as the light reception unit acquiring the pulse wave detection signal which is a signal originally planned to be detected, the first light reception unit 141 is used.

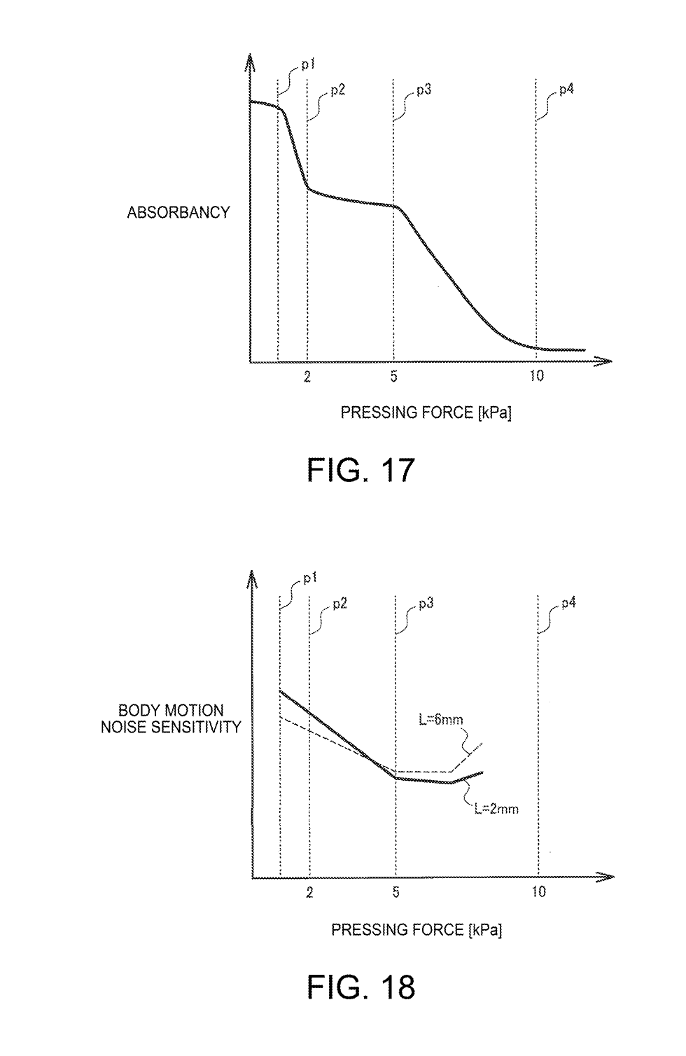

In addition, as described later in FIG. 17 or the like, when the height of the light transmissive member 50 is different, a pressing force which is applied to the skin by the light transmissive member 50 at the time of mounting the biological information detecting device is changed. The pressing force increases as the height of the light transmissive member 50 becomes higher, and a blood capillary on an upper layer of the subcutaneous tissue is pressed by the pressing force. Since blood flow through the blood capillary of the upper layer is easily affected by the body motion, the blood flow is suppressed by pressing the blood capillary on the upper layer, and thus it is possible to decrease sensitivity of the body motion noise. For this reason, as the light reception unit which acquires the pulse wave detection signal, the first light reception unit 141 disposed under the convex portion 52-1 is used, and as the light reception unit which acquires the body motion detection signal, the second light reception unit 142 disposed under the convex portion 52-2 is used.

### 9. Distance Between Light Emission Unit and Light Reception Unit

Next, an influence of the distance between the light emission unit and the light reception unit on the detection signal will be described.

FIG. 15 is a diagram for illustrating an influence of the distance between the light emission unit and the light reception unit on a penetration depth of the light. The light emission unit 150, the first light reception unit 141, and the second light reception unit 142 are in contact with a skin surface Sf of a wrist of the user. In practice, the light transmissive member 50 is in contact with the skin surface Sf, and for the sake of simple description, the light transmissive member 50 is omitted from FIG. 15.

It is found that sensitivity with respect to a deep portion in a living body relatively decreases compared to sensitivity with respect to a shallow portion as the distance between the light emission unit and the light reception unit becomes shorter. That is, the intensity of the light which is emitted from the light emission unit 150, is reflected on a position of a depth D1 in a body tissue, and reaches the first light reception unit 141 is stronger than the intensity of the light which is emitted from the light emission unit 150, is reflected on a position of a depth D2 deeper than the depth D1, and reaches the first light reception unit 141. On the other hand, the intensity of the light which is emitted from the light emission unit 150, is reflected on the position of the depth D1, and reaches the second light reception unit 142 is stronger than the intensity of the light which is emitted from the light emission unit 150, is reflected on the position of the depth D2, and reaches the second light reception unit 142, but there is no difference in the occurrence of the light in the first light reception unit 141. For this reason, the first light reception unit 141 is suitable for measuring the pulse wave in the blood vessel which is in a relatively shallow position compared to the second light reception unit 142.

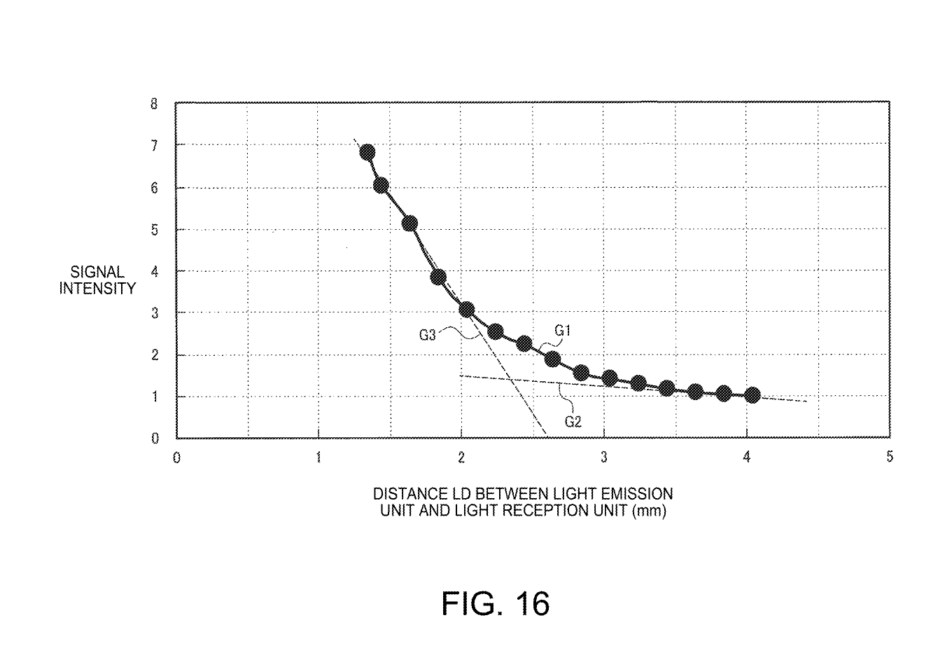

FIG. 16 is a diagram illustrating a relationship between distance LD between the light emission unit 150 and the light reception unit and signal intensity. As it is obvious from FIG. 16, a signal intensity of the detection signal increases as the distance LD between the light emission unit 150 and the light reception unit becomes shorter, and thus detection performance such as sensitivity is improved. Accordingly, it is preferable for the first light reception unit 141 which mainly detects the pulse signal as the distance LD with respect to the light emission unit 150 to become shorter.

For example, as it is obvious from a tangential line G2 on a side on which the distance increases, in a characteristic curve G1 of FIG. 16, the characteristic curve G1 is saturated in a range of LD≧3 mm. In contrast, the signal intensity considerably increases as the distance LD becomes shorter in a range of LD<3 mm. Accordingly, in this sense, it is preferable to satisfy LD<3 mm.

In addition, the distance LD has a lower limit value, and it is not preferable that the distance LD becomes excessively shorter. When a distance which is able to be measured in a depth direction from the skin surface Sf is LB, a relationship of LB=LD/2 is generally established. For example, a depth of 100 μm to 150 μm from the skin surface Sf does not reach the shallowest blood capillary of the surface skin, and thus is not a detection target of the pulse wave. For this reason, when LD≦2×LB=2×100 μm to 2×150 μm)=0.2 mm to 0.3 mm is satisfied, it is expected that the detection signal of the pulse wave decreases extremely. That is, the detection performance is improved as the distance LD becomes shorter, but there is a limitation, and the distance LD has a lower limit value. In this embodiment, it is necessary that the first light reception unit 141 detects the pulse signal with sufficient intensity, and thus it is preferable to satisfy 1.0 mm≦L1≦3.0 mm.

In contrast, the distance L2 between the light emission unit 150 and the second light reception unit 142 may be set such that sensitivity with respect to the pulse signal decreases and sensitivity with respect to the body motion noise increases compared to the first light reception unit 141. For example, when L2<1.0 mm or 3.0 mm<L2 is satisfied, the degree of the pulse signal decreases and the degree of the body motion noise increases (an MN ratio decreases) compared to the first light reception unit 141 in which 1.0 mm≦L1≦3.0 mm is satisfied.

However, in the second light reception unit 142, an MN ratio of the detection signal (M indicates the pulse signal, N indicates the noise, and the MN ratio is a ratio of the pulse signal and the noise (a general SN ratio)) may sufficiently decrease compared to the MN ratio of the detection signal of the first light reception unit 141. That is, from a point that the distance is set as an absolute value of L2<1.0 mm or 3.0 mm<L2, importance may be placed on a point that the value of L2 with respect to L1 is changed such that a certain degree of difference (for example, a degree of enabling the noise reduction processing to be performed by the spectral subtraction described later) is able to occur between the first detection signal and the second detection signal.

That is, it is sufficient that the MN ratio of the second detection signal from the second light reception unit 142 decreases compared to the first detection signal, and thus a certain degree of pulse component may not be prevented from being included, in other words, L2 may be in a range of 1.0 mm≦L2≦3.0 mm.

Here, as a relationship between L1 and L2 for allowing a difference to occur between the first detection signal and the second detection signal, for example, L2>2×L1 or the like may be used. In this case, when L1=1.0 mm is satisfied, L2>2.0 mm is satisfied. The pulse signal is detected with a certain degree of intensity, and it is possible to satisfy a condition that the MN ratio of the second detection signal decreases compared to the first detection signal in which L1 shorter than L1 is set.

### 10. Pressing Force of Light Transmissive Member

Next, an influence of the pressing force of the light transmissive member on the detection signal will be described.

FIG. 17 is a diagram exemplifying a change in absorbancy with respect to the pressing force. A horizontal axis indicates a pressing force, and a vertical axis indicates absorbancy. When the pressing force is changed, the blood vessel which is subjected to the influence is changed. The blood vessel which is most easily subjected to the influence, that is, which is subjected to the influence at the lowest pressing force is a blood capillary. In an example of FIG. 17, the amount of change in the absorbancy increases at a point where the pressing force exceeds p1, and this indicates that the blood capillary is starting to collapse by the pressing force. When the pressing force exceeds p2, the change in the absorbancy becomes smooth, and this indicates that the blood capillary approximately completely collapses (is closed). The blood vessel which is easily subjected to the influence next to the blood capillary is an artery. Further, when the pressing force increases and exceeds p3, the amount of change in the absorbancy increases again, and this indicates that the artery is starting to collapse by the pressing force. When the pressing force exceeds p4, the change in the absorbancy becomes smooth, and this indicates that the artery approximately completely collapses (is closed).

FIG. 18 is a diagram exemplifying a change in body motion noise sensitivity with respect to the pressing force. In FIG. 18, an example where the distance L between the light emission unit and the light reception unit is 2 mm and an example where the distance L between the light emission unit and the light reception unit is 6 mm are illustrated together. Any of the examples where the distance L is 2 mm and 6 mm shows a trend in which the noise sensitivity increases as the pressing force becomes lower, and the noise sensitivity decreases as the pressing force becomes higher. It is considered that this is because blood flowing in the blood capillary is easily moved by the body motion, and thus the noise due to the body motion is easily included in the light reflected on the blood capillary in a comparatively shallow position of the body tissue.

In this embodiment, at the time of measuring the biological information of the subject, when the pressing force in the position or the region corresponding to the first light reception unit 141 of the light transmissive member 50 is p1, and the pressing force in the position or the region corresponding to the second light reception unit 142 of the light transmissive member 50 is p2, p1>p2 is set. A difference in the pressing force is realized by a difference in the height of the light transmissive member 50 which is in contact with the subject.

Specifically, the second light reception unit 142 increases the ratio of the body motion noise by detecting the signal corresponding to the blood capillary, and the first light reception unit 141 increases the ratio of the pulse signal by measuring the signal (the pulse signal) corresponding to the artery. That is, the pressing force in the second light reception unit 142 is designed to be in a range of p1 to p2 (a pressure at which the blood capillary does not completely collapse), and the pressing force in the first light reception unit 141 is designed to be in a range of p3 to p4 (a pressure at which the blood capillary collapses). For example, it is preferable that a difference in the pressing force between the first light reception unit 141 and the second light reception unit 142 is greater than or equal to 2.0 kPa and less than or equal to 8.0 kPa.

### 11. Body Motion Noise Reduction Processing (Spectral Subtraction)

Next, the body motion noise reduction processing performed by the processing unit 200 will be described. In the body motion noise reduction processing, the spectral subtraction performed on the basis of the second detection signal, and the adaptive filter processing performed on the basis of the motion sensor unit 170 are included.

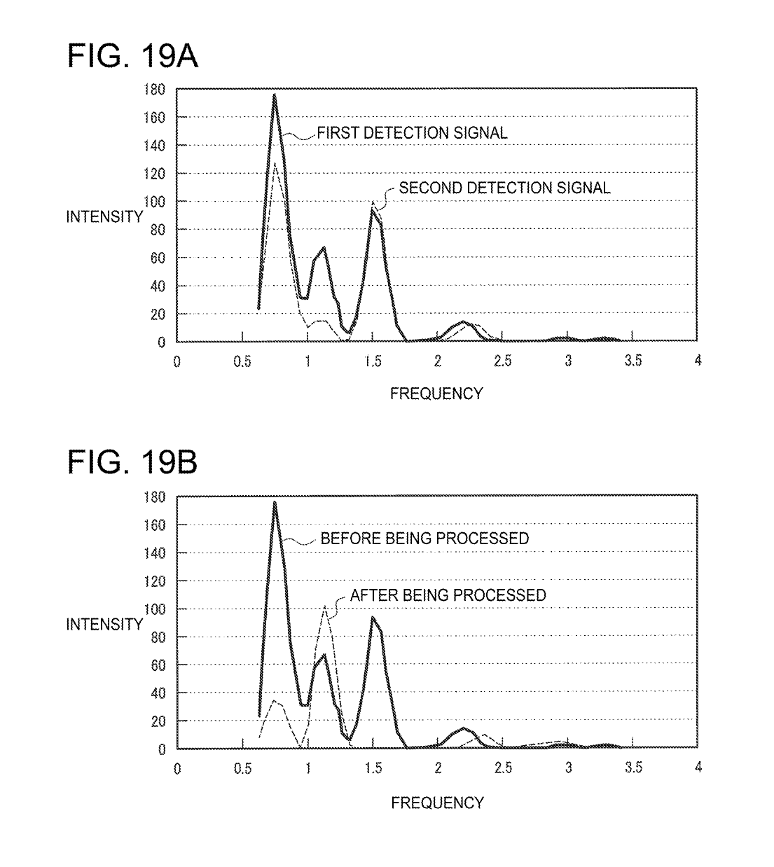

First, the spectral subtraction will be described. FIGS. 19A and 19B are diagrams illustrating the noise reduction processing of the first detection signal which is performed on the basis of the second detection signal by using the spectral subtraction. In the spectral subtraction, the frequency conversion processing is performed with respect to each of the first detection signal and the second detection signal, and thus a spectrum is obtained. Then, a noise spectrum is estimated from the spectrum of the second detection signal, and the estimated noise spectrum is subtracted from the spectrum of the first detection signal.

In FIG. 19A, the spectrum of the first detection signal and the spectrum of the second detection signal which are actually obtained are illustrated. As described above, by using the biological information detecting device according to this embodiment, the spectrum of the second detection signal becomes a spectrum which mainly corresponds to the noise component. That is, it is possible to estimate that a frequency at which a large peak appears in the spectrum of the second detection signal is a frequency corresponding to the body motion noise. In practice, only the peak may be subtracted from the spectrum of the second detection signal, but the configuration is not limited thereto, and for example, the entire spectrum of the second detection signal may be subtracted from the entire spectrum of the first detection signal.

In the subtraction, in order to cancel out the noise, for example, one of the first detection signal and the second detection signal is multiplied by a coefficient. The coefficient, for example, is obtained from the signal intensity of a predetermined frequency. Alternatively, for example, the noise may be separated from the signal by a method such as clustering, and the coefficient may be calculated such that the noise of the first detection signal and the noise of the second detection signal have the same intensity.

An example of the first detection signal before and after the body motion noise reduction processing of the spectral subtraction is illustrated in FIG. 19B. As it is obvious from FIG. 19B, the body motion noise which appears in 0.7 Hz to 0.8 Hz (42 to 48 in the number of pulses) and 1.5 Hz (90 in the number of pulses) is suppressed to be small by the body motion noise reduction processing, and a probability of erroneously determining the noise as the pulse signal is able to be suppressed. On the other hand, the signal level is able to be maintained without reducing the spectrum corresponding to the pulse signal which appears before and after 1.1 Hz (66 in the number of pulses).

The spectral subtraction is realized by the frequency conversion processing such as Fast Fourier Transform (FFT), and the subtraction processing in the spectrum, and thus has an advantage of having a simple algorithm and a small calculation amount. In addition, there is no learning element as in the adaptive filter processing described later, and thus the spectral subtraction has properties of high instant responsiveness.

### 12. Body Motion Noise Reduction Processing (Adaptive Filter Processing)

Next, the body motion noise reduction processing performed by using the adaptive filter processing on the basis of the detection signal from the motion sensor will be described.

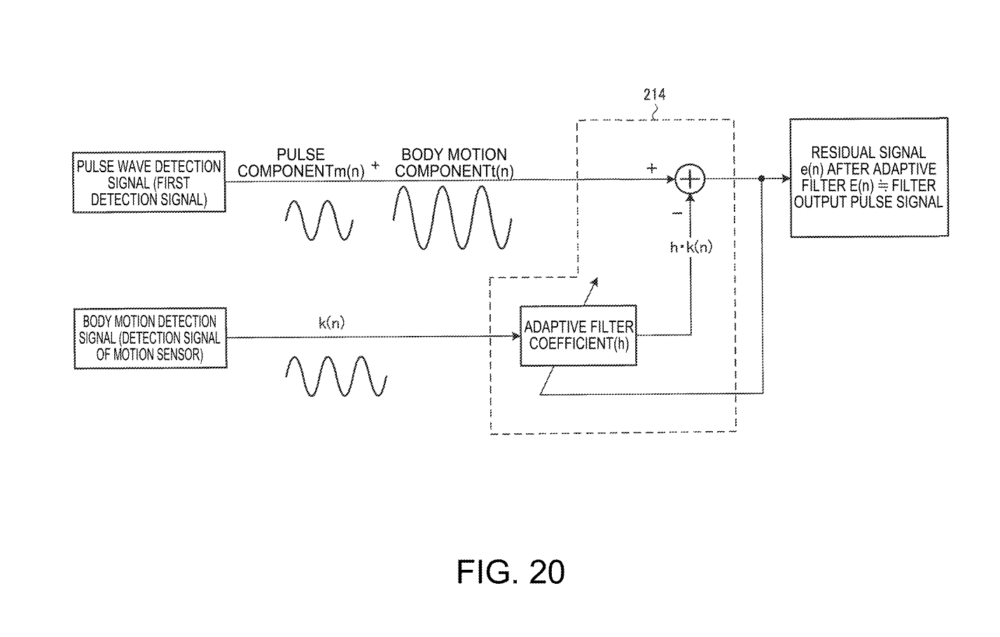

In FIG. 20, a specific example of the noise reduction processing using an adaptive filter 214 is illustrated. The detection signal of the motion sensor unit 170 corresponds to the body motion noise, and thus the noise component specified from the detection signal is subtracted from the first detection signal, and the basic configuration is identical to that of the spectral subtraction.

However, even though both of the body motion noise in the pulse wave detection signal and the body motion detection signal from the body motion sensor are signals due to the same body motion, the signal levels thereof are not identical to each other. Accordingly, the filter processing in which a filter coefficient is adaptively determined is performed with respect to the body motion detection signal, and thus an estimated body motion noise component is calculated, and a difference between the pulse wave detection signal and the estimated body motion noise component is obtained. The filter coefficient is adaptively (by performing learning) determined, and thus it is possible to improve precision of the noise reduction processing, but it is necessary to consider a processing load in the determination of the filter coefficient or delay of output. Furthermore, the adaptive filter processing is a widely known method, and thus the specific description thereof will be omitted.

By combining the adaptive filter processing using the motion sensor with the spectral subtraction using the second detection signal, it is possible to more precisely reduce the body motion noise compared to a case where only the spectral subtraction is performed. For example, in FIG. 19B, the noise in 0.7 Hz to 0.8 Hz or 2.3 Hz to 2.4 Hz is not able to be reduced, but by combining the processing using the detection signal from the motion sensor, the noise is able to be reduced.

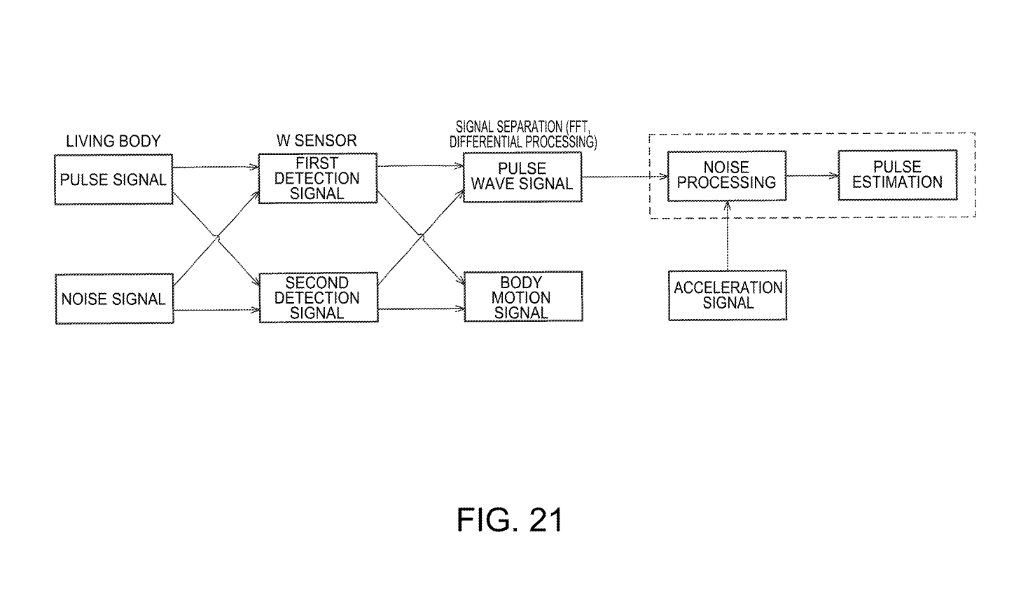

In this embodiment, the body motion noise reduction processing of the spectral subtraction is performed, and the adaptive filter processing using the motion sensor is performed with respect to the signal after being subjected to the processing. In this case, a flow of each signal is illustrated in FIG. 21.

As illustrated in FIG. 21, the pulse signal and the noise signal are able to be detected from the living body, and each detection signal from a plurality of light reception units includes both of the pulse signal and the noise signal. However, in this embodiment, the ratio is changed for each light reception unit, in the first detection signal, the amount of pulse signal is comparatively large, and in the second detection signal, the ratio of the pulse signal is lower than that in the first detection signal (the ratio of the body motion noise is high). Then, the pulse signal is separated from the body motion signal (the body motion noise) by using these two detection signals. The processing is realized by the spectral subtraction described above. Then, the second body motion noise reduction processing using the detection signal of the motion sensor (in FIG. 21, the acceleration signal) is performed with respect to the separated pulse signal (the first detection signal after being subjected to the body motion noise reduction processing), and from the result thereof, the number of pulses or the like is estimated.

Furthermore, as described above, this embodiment is specifically described, but a person skilled in the art is able to easily understand that the embodiment is able to be modified without substantially departing from the new matters and the effects of the invention. Accordingly, all of these modification examples are included in the range of the invention. For example, in the specification and the drawings, terms which are stated at least once along with different terms having a more extended meaning or the same meaning, are able to be replaced by the different terms in any portion of the specification and the drawings. In addition, the configuration and the operation of the biological information detecting device and the like are not limited to that described in this embodiment, and are able to be variously modified.

In the embodiment of the invention, the detection operation is controlled on the basis of the sleep state of the subject, but the configuration is not limited thereto. For example, the activity situation of the subject is determined on the basis of the signal from the acceleration sensor 172, and when it is determined that the noise due to the body motion decreases during reading, desk work, or the like, the first detection operation of the first light reception unit and the second detection operation of the second light reception unit may be controlled. According to such a configuration, it is possible to reduce electricity consumption not only during sleep but also during the activities of the daily life.

What is claimed is:

1. A biological information detecting device, comprising:
a first light reception unit which receives light from a subject;
a second light reception unit which receives light from the subject;
at least one light emission unit which emits light to the subject; and
a processing unit,
wherein the processing unit determines an active state of the subject on the basis of a first detection signal detected by the first light reception unit and a second detection signal detected by the second light reception unit, and controls a first detection operation of the light emission unit and the first light reception unit and a second detection operation of the light emission unit and the second light reception unit according to the active state.

2. The biological information detecting device according to claim 1,
wherein when a distance between the light emission unit and the first light reception unit is L1, and a distance between the light emission unit and the second light reception unit is L2, L1<L2 is satisfied.

3. The biological information detecting device according to claim 1,
wherein the active state is a sleep state of the subject, and
the processing unit sets the second detection operation to be in a normal operation mode when it is determined that the subject is in a first sleep state, and sets the second detection operation to be in a non-operation mode when it is determined that the subject is in a second sleep state which is deeper than the first sleep state.

4. The biological information detecting device according to claim 3,
wherein the processing unit allows the light emission unit to emit light during a first period of detecting the light from the subject by the first light reception unit and a second period of detecting the light from the subject by the second light reception unit in the normal operation mode of the second detection operation, and stops light emission of the light emission unit during the second period in the non-operation mode of the second detection operation.

5. The biological information detecting device according to claim 3,
wherein the first sleep state is REM sleep, and
the second sleep state is non-REM sleep.

6. The biological information detecting device according to claim 1,
wherein the processing unit sets the second detection operation to be in a normal operation mode when it is determined that the subject is in an awake state, and sets the second detection operation to be in a non-operation mode when it is determined that the subject is in a predetermined sleep state.

7. The biological information detecting device according to claim 1,
wherein the processing unit performs body motion noise reduction processing which reduces a body motion noise of the first detection signal on the basis of the second detection signal, and calculates biological information on the basis of the first detection signal after being subjected to the body motion noise reduction processing.

8. The biological information detecting device according to claim 7,
wherein the processing unit obtains pulse wave information as the biological information, and determines the active state on the basis of the pulse wave information.

9. The biological information detecting device according to claim 8,
wherein the processing unit obtains a first index indicating activity of a sympathetic nerve and a second index indicating activity of a parasympathetic nerve by frequency analysis of the pulse wave information, and determines the active state on the basis of the first index and the second index.

10. The biological information detecting device according to claim 7, further comprising:
a motion sensor unit which detects body motion information of the subject,
wherein the processing unit determines the active state on the basis of the body motion information.

11. The biological information detecting device according to claim 10,
wherein the processing unit sets the motion sensor unit to be in a low power consumption mode when it is determined that the subject has transitioned from the awake state to the sleep state.

12. A biological information detecting device, comprising:
a first light reception unit which receives light from a subject;
a second light reception unit which receives light from the subject;
at least one light emission unit which emits light to the subject;
a substrate on which at least the first light reception unit and the light emission unit are arranged;
a light transmissive member which is disposed in a position on the subject side from the first light reception unit side and the second light reception unit side, transmits the light from the subject, and is in contact with the subject at the time of measuring biological information of the subject; and
a processing unit;
wherein in a plan view of a direction from the biological information detecting device to the subject, when a distance between the substrate and a surface of the light transmissive member in contact with the subject in a region in which the light transmissive member and the first light reception unit overlap each other is h1, and a distance between the substrate and a surface of the light transmissive member in contact with the subject in a region in which the light transmissive member and the second light reception unit overlap each other is h2, h1>h2 is satisfied, and
the processing unit determines an active state of the subject on the basis of a first detection signal detected by the first light reception unit and a second detection signal detected by the second light reception unit, and controls a first detection operation of the light emission unit and the first light reception unit and a second detection operation of the light emission unit and the second light reception unit according to the active state.
